(19) United States
(12) Patent Application Publication
(10) Pub. No.: US 2018/0133270 A1
(43) Pub. Date: May 17, 2018

(54) RECOMBINANT ONCOLYTIC VIRUSES AND USES THEREOF

(71) Applicant: BRITISH COLUMBIA CANCER AGENCY BRANCH, Vancouver, British Columbia (CA)

(72) Inventors: Brad NELSON, Victoria, British Columbia (CA); Kwame TWUMASl-BOATENG, Richmond, British Columbia (CA); David KROEGER, Victoria, British Columbia (CA); Parv CHAPANI, Vancouver, British Columbia (CA); Dakota PEACOCK, Kelowna, British Columbia (CA)

(21) Appl. No.: 15/574,685

(22) PCT Filed: May 19, 2016
(86) PCT No.: PCT/IB2016/052922

§ 371 Date: Nov. 16, 2017

Related U.S. Application Data
(60) Provisional application No. 62/163,918, filed on May 19, 2015.

(51) Int. Cl. A61K 35/768; C12N 15/86; A61P 35/00

(52) U.S. Cl. CPC A61K 35/768; C07K 14/52; A61P 35/00; C12N 15/86

(57) ABSTRACT

The present invention relates to recombinant oncolytic viruses. More specifically, the present invention relates to recombinant oncolytic viruses expressing a heterologous B cell attractant polypeptide or a T cell attractant polypeptide.

### FIELD OF INVENTION

The present invention relates to recombinant oncolytic viruses. More specifically, the present invention relates to recombinant oncolytic viruses expressing a heterologous B cell attractant polypeptide or a T cell attractant polypeptide.

### BACKGROUND OF THE INVENTION

The immune system is thought to play a key role in clinical outcomes in cancer patients. Tumour infiltrating lymphocytes (TIL) are implicated in the body's defense against cancer. For example, CD8+ tumour-infiltrating T cells have been associated with markedly increased survival in ER-breast cancer, as well as ovarian cancer (13, 23). Furthermore, tumour-infiltrating B cells have been implicated as a positive prognostic factor in ovarian cancer (16).

In addition to the sheer number of TIL in a tumour, the organization of lymphocytes within the tumour is thought to play an important role in the anti-tumour immune response. Tertiary Lymphoid Structures (TLS), which are in situ aggregates of immune cells resembling secondary lymphoid organs, have been correlated with increased patient survival in a number of cancers (reviewed in 6, 20), including breast, colorectal, and other human cancers.

The chemokines CXCL10 and CXCL13 have been associated with TLS (4, 10, 11, 15). CXCL10 expression can be induced by type I or type II interferons produced from processes such as viral infection or antigen-specific activation of T cells. CXCL10 then acts as a chemoattractant for activated T cells. CXCL13 is a chemoattractant for B cells and T follicular Helper cells (T_{FH}). Moreover, endoscopic injection of recombinant of CXCL13 in a mouse model of colorectal cancer resulted in tumour rejection in 80% of treated mice (1).

Oncolytic viruses (OVs) are viruses that selectively replicate in cancer cells. Live replicating OVs have been tested in clinical trials in a variety of human cancers (reviewed in 17). OVs can induce anti-tumour immune responses, as well as direct lysis of tumour cells. Common OVs include attenuated strains of Vesicular Stomatitis Virus (VSV) and Vaccinia Virus (VV).

### SUMMARY OF THE INVENTION

The present invention relates to recombinant oncolytic viruses. More specifically, the present invention relates to recombinant oncolytic viruses expressing a heterologous B cell attractant polypeptide or a T cell attractant polypeptide.

In one aspect, the present invention provides a recombinant oncolytic virus including a heterologous nucleic acid sequence encoding a B cell attractant polypeptide or a T cell attractant polypeptide, where the heterologous nucleic acid sequence is stably incorporated into the genome of the recombinant oncolytic virus. The recombinant oncolytic virus may be attenuated. The recombinant oncolytic virus may be an oncolytic RNA virus or an oncolytic DNA virus.

In some embodiments, the recombinant oncolytic virus may be an oncolytic RNA virus, such as a vesicular stomatitis virus (VSV), Maraba Virus, Newcastle Disease Virus, Poliovirus, Measles Virus or Reovirus, and the heterologous nucleic acid sequence may encode a B cell attractant polypeptide, such as a CXCL12 or CXCL13 polypeptide.

In some embodiments, the recombinant oncolytic virus may be an oncolytic DNA virus, such as a Vaccinia Virus (VV), Herpes Simplex Virus (HSV), or Adenovirus, and the heterologous nucleic acid sequence may encode a T cell attractant polypeptide, such as CXCL10.

In some embodiments, the recombinant oncolytic virus may be VSV-CXCL12, VV-CXCL12, VSV-CXCL13, VV-CXCL13, VSV-CXCL10 or VV-CXCL10.

In some aspects, the present invention provides a pharmaceutical composition including a recombinant oncolytic virus, as described herein, and a pharmaceutically acceptable carrier. The pharmaceutical composition may include a VSV-CXCL13 in combination with a VV-CXCL12, a VV-CXCL13 or a VV-CXCL10. The pharmaceutical composition may be formulated for systemic administration.

In some aspects, the present invention provides a method of treating a cancer by administering a therapeutically effective amount of the recombinant oncolytic virus, or a pharmaceutical composition, as described herein, to a subject in need thereof. The cancer may be a breast cancer, colorectal cancer, lung cancer, melanoma, or ovarian cancer. In alternative aspects, the present invention provides a recombinant oncolytic virus, or a pharmaceutical composition, as described herein, for treating a cancer in a subject in need thereof.

In some aspects, the present invention provides a method of recruiting immune cells to a tumour by contacting the tumour with the recombinant oncolytic virus, as described herein.

In some aspects, the present invention provides a method of inhibiting the growth or promoting the killing of a tumour cell, by contacting the tumour cell with a recombinant oncolytic virus, as described herein. The recombinant oncolytic virus may be provided at a dosage sufficient to cause cell death of the tumor cell.

This summary does not necessarily describe all features of the invention.

### BRIEF DESCRIPTION OF THE DRAWINGS

These and other features of the invention will become more apparent from the following description in which reference is made to the appended drawings wherein:

FIG. 1A is a graph showing the verification of chemokine CXCL10 production by recombinant oncolytic vesicular stomatitis virus (VSV) and vaccinia virus (VV).

FIG. 1B is a graph showing the verification of chemokine CXCL13 production by recombinant oncolytic vesicular stomatitis virus (VSV) and vaccinia virus (VV).

FIG. 2A is a schematic diagram showing the experimental approach to determine immune cell recruitment and cluster formation by VSV-CXCL13 in mouse mammary tumour cells.

FIG. 2B is a graph showing the number of B cell-containing lymphoid clusters in mouse mammary tumour cells after intratumoural injection of PBS, VSV-GFP and VSV-CXCL13.

FIG. 3A is a schematic diagram showing the experimental approach to determine therapeutic efficacy of VSV-CXCL13 in a mouse model of mammary cancer.

FIG. 3B is a graph showing tumour size in response to intratumoural PBS.

FIG. 3C is a graph showing tumour size in response to intratumoural VSV-GFP.

FIG. 3D is a graph showing tumour size in response to intratumoural VSV-CXCL13.

FIG. 3E is a graph comparing to the survival of mice treated with either intratumoural PBS, VSV-GFP or VSV-CXCL13.

FIG. 4A is a schematic diagram showing the experimental approach to determine therapeutic efficacy of VSV-CXCL10 in mouse mammary tumour cells.

FIG. 4B is a graph showing tumour size in response to intratumoural PBS.

FIG. 4C is a graph showing tumour size in response to intratumoural VSV-GFP.

FIG. 4D is a graph showing tumour size in response to intratumoural VSV-CXCL10.

FIG. 4E is a graph comparing the survival of mice treated with either intratumoural PBS, VSV-GFP or VSV-CXCL10.

FIG. 5A shows the nucleotide sequence of a murine CXCL10 lacking the 3′ UTR (SEQ ID NO: 1). This sequence was cloned into the VSV-d51 plasmid to generate VSV-CXCL10.

FIG. 5B shows the amino acid sequence of a murine CXCL10 (SEQ ID NO: 2).

FIG. 5C shows the nucleotide sequence of a human CXCL10 cDNA, NCBI Reference Sequence: NM_001565.1 (SEQ ID NO: 3).

FIG. 5D shows the amino acid sequence of a human CXCL10, NCBI Reference Sequence: NP_001556.2 (SEQ ID NO: 4).

FIG. 5E shows the nucleotide sequence of a murine CXCL13 lacking the 3′ UTR (SEQ ID NO: 5). This sequence was cloned into the VSV-d51 plasmid to generate VSV-CXCL13.

FIG. 5F shows the amino acid sequence of a murine CXCL13 (SEQ ID NO: 6).

FIG. 5G shows the nucleotide sequence of a human CXCL13 cDNA, NCBI Reference Sequence: NM_006419.2 (SEQ ID NO: 7).

FIG. 5H shows the amino acid sequence of a human CXCL13, NCBI Reference Sequence: NP_006410.1 (SEQ ID NO: 8).

FIG. 5I shows the nucleotide sequence of a murine CXCL12 cDNA (SEQ ID NO: 9).

FIG. 5J shows the amino acid sequence of a murine CXCL12, (SEQ ID NO: 10).

FIG. 5K shows the nucleotide sequence of a human CXCL12 variant 2 cDNA, NCBI Reference Sequence: NM_000609.6 (SEQ ID NO: 11).

FIG. 5L shows the nucleotide sequence of a human CXCL12 variant 1 cDNA, NCBI Reference Sequence: NM_000609.3 (SEQ ID NO: 12).

FIG. 5M shows the amino acid sequence of a human CXCL12-beta polypeptide, NCBI Reference Sequence: NP_000600.1 (SEQ ID NO: 13).

FIG. 5N shows the amino acid sequence of a human CXCL12-alpha polypeptide, NCBI Reference Sequence: NP_954637.1 (SEQ ID NO: 14).

FIG. 5O shows the amino acid sequence of a human CXCL12-gamma polypeptide, NCBI Reference Sequence: NP_001029058.1 (SEQ ID NO: 15).

FIG. 5P shows the amino acid sequence of a human CXCL12-delta polypeptide, NCBI Reference Sequence: NP_001171605.1 (SEQ ID NO: 16).

FIG. 5Q shows the amino acid sequence of a human CXCL12-isoform 5 polypeptide, NCBI Reference Sequence: NP_001264919.1 (SEQ ID NO: 17).

### DETAILED DESCRIPTION

The present disclosure relates to recombinant oncolytic viruses. More specifically, the present disclosure relates in part to recombinant oncolytic viruses expressing a heterologous B cell and/or T cell attractant polypeptide, and uses thereof.

B Cell Attractant Polypeptide

A “B cell attractant polypeptide,” as used herein, refers to a polypeptide that is capable of recruiting B cells to a particular location. In some embodiments, the location may be a location capable of supporting the replication of an oncolytic virus. In some embodiments, the location may be a solid tumour.

In some embodiments, a B cell attractant polypeptide encoded by an oncolytic virus may increase the total number of B cells in a particular location, such as a solid tumour, by at least 10%, 15%, 20%, 25%, 30%, 35%, 40%, 45%, 50%, 55%, 60%, 65%, 70%, 75%, 80%, 85%, 90%, 95%, 100%, or more, compared to the number of B cells in that particular location in the absence of the B cell attractant polypeptide. In some embodiments, a B cell attractant polypeptide encoded by an oncolytic virus may increase the total number of B cells in a particular location, such as a solid tumour, by at least 1 fold, 2 fold, 3 fold, 4 fold, 5 fold, or more, compared to the number of B cells in that particular location in the absence of the B cell attractant polypeptide. In some embodiments, a B cell attractant polypeptide, as disclosed herein, may induce the formation of clusters of B cells (a “B cell cluster”) in a particular location, such as a solid tumour. A “B cell cluster,” as used herein, refers to aggregates of lymphoid cells, primarily B cells, in a particular location, such as a solid tumour. It is to be understood that, in some embodiments, a B cell cluster may include small numbers of T cells or other cells. In some embodiments, a B cell cluster may include fewer than 10% T cells. In some embodiments, a B cell cluster may lack the characteristics, for example the structural organization, of a Tertiary Lymphoid Structure (TLS). Accordingly, in some embodiments, a B cell attractant polypeptide, as disclosed herein, may induce the formation of a B cell cluster but not a TLS. The presence of a B cell cluster may be determined by using, for example, immunohistochemical techniques and determining the presence of immune cells, such as T cells or B cells in a location, such as a solid tumour. In some embodiments, the presence of a B cell cluster may be determined by comparing a sample, such as solid tumour sample, that may or not have been exposed to a B cell attractant polypeptide.

In some embodiments, a B cell attractant polypeptide, as disclosed herein, may be a biologically active fragment. By “biologically active fragment,” as used herein, is meant a portion of a B cell attractant polypeptide that is shorter than the full length polypeptide by one or more residues and is capable of recruiting B cells to a particular location, such as a solid tumour.

A B cell attractant polypeptide, as disclosed herein, may be a chemokine, such as a homeostatic chemokine. In some embodiments, a B cell attractant polypeptide, as disclosed herein, may be CXCL12 or CXCL13, or a biologically active fragment thereof. In some embodiments, a B cell attractant polypeptide, as disclosed herein, may include without limitation, a polypeptide having a sequence substantially identical to a CXCL12 or CXCL13 sequence.

In some embodiments, a CXCL12 polypeptide may have a sequence as set forth in or substantially identical to one or more of SEQ ID NOs. 10, 13-17, or one or more of the sequences set forth in MGI OTTMUSP00000026114 or NCBI Reference numbers NP_000600.1, NP_954637.1, NP_001029058.1, NP_001171605.1 or NP_001264919.1.

In alternative embodiments, a CXCL12 polypeptide may have a sequence encoded by one or more of SEQ ID NOs. 9, 11 or 12, or set forth in MGI OTTMUST00000054664 or NCBI Reference numbers NM_000609.6 or NM_000609.3, or a sequence substantially identical thereto.

In alternative embodiments, a CXCL12 nucleic acid molecule may have a sequence as set forth in, or substantially identical to, one or more of the nucleic acid sequences set forth in SEQ ID NOs. 9, 11 or 12, or in MGI OTTMUST00000054664 or NCBI Reference numbers NM_000609.6 or NM_000609.3, or a fragment thereof, for example, a cDNA fragment lacking the 3′ UTR.

In some embodiments, a CXCL13 polypeptide may have the sequence as set forth in SEQ ID NOs. 6 or 8, or set forth in MGI OTTMUSP00000072614 or NCBI Reference number NP_006410.1, or a sequence substantially identical thereto.

In alternative embodiments, a CXCL13 polypeptide may have a sequence encoded by, or substantially identical to, the one or more of the sequences as set forth in SEQ ID NOs. 5 or 7, or set forth in OTTMUST00000138021 or NCBI Reference number NMhd —006419.2, or a fragment thereof, for example, a cDNA fragment lacking the 3′ UTR.

In alternative embodiments, a CXCL13 nucleic acid molecule may have a sequence encoded by, or substantially identical to, one or more of the nucleic acid sequences as set forth in SEQ ID NOs. 5 or 7, or set forth in OTTMUST00000138021 or NCBI Reference number NM_006419.2, or a fragment thereof, for example, a cDNA fragment lacking the 3′ UTR.

T Cell Attractant Polypeptides

A “T cell attractant polypeptide,” as used herein, refers to a polypeptide that is capable of recruiting T cells to a particular location. In some embodiments, the location may be a location capable of supporting the replication of an oncolytic virus. In some embodiments, the location may be a solid tumour.

In some embodiments, a T cell attractant polypeptide encoded by an oncolytic virus may increase the total number of T cells in a particular location, such as a solid tumour, by at least 10%, 15%, 20%, 25%, 30%, 35%, 40%, 45%, 50%, 55%, 60%, 65%, 70%, 75%, 80%, 85%, 90%, 95%, 100%, or more, compared to the number of T cells in that particular location in the absence of the T cell attractant polypeptide. In some embodiments, a T cell attractant polypeptide encoded by an oncolytic virus may increase the total number of T cells in a particular location, such as a solid tumour, by at least 1 fold, 2 fold, 3 fold, 4 fold, 5 fold, or more, compared to the number of T cells in that particular location in the absence of the T cell attractant polypeptide. In some embodiments, a T cell attractant polypeptide, as disclosed herein, may induce the formation of clusters of B cells (a “T cell cluster”) in a particular location, such as a solid tumour. A “T cell cluster,” as used herein, refers to aggregates of lymphoid cells, primarily T cells, in a particular location, such as a solid tumour. It is to be understood that, in some embodiments, a T cell cluster may include small numbers of B cells or other cells. In some embodiments, a T cell cluster may include fewer than 10% B cells. In some embodiments, a T cell cluster may lack the characteristics, for example the structural organization, of a Tertiary Lymphoid Structure (TLS). Accordingly, in some embodiments, a T cell attractant polypeptide, as disclosed herein, may induce the formation of a T cell cluster but not a TLS. The presence of absence of a T cell cluster may be determined by using, for example, immunohistochemical techniques and determining the presence of immune cells, such as T cells or B cells in a location, such as a solid tumour. In some embodiments, the presence of a T cell cluster may be determined by comparing a sample, such as solid tumour sample, that may or not have been exposed to a T cell attractant polypeptide.

In some embodiments, a T cell attractant polypeptide, as disclosed herein, may be a biologically active fragment. By “biologically active fragment,” as used herein, is meant a portion of a T cell attractant polypeptide that is shorter than the full length polypeptide by one or more residues and is capable of recruiting T cells to a particular location, such as a solid tumour.

A T cell attractant polypeptide, as disclosed herein, may be a chemokine, such as a CXCL10 polypeptide.

In some embodiments, a CXCL10 polypeptide may have the sequence as set forth in SEQ ID NOs. 2 or 4, or set forth in OTTMUSP00000036424 or

NCBI Reference number NP_001556.2, or a sequence substantially identical thereto.

In alternative embodiments, a CXCL10 polypeptide may have a sequence encoded by, or substantially identical to, the one or more of the sequences as set forth in SEQ ID NOs. 1 or 3, or set forth in OTTMUSG00000028740 or NCBI Reference number NM_001565.1, or a fragment thereof, for example, a cDNA fragment lacking the 3′ UTR.

In alternative embodiments, a CXCL10 nucleic acid molecule may have a sequence encoded by, or substantially identical to, one or more of the nucleic acid sequences as set forth in SEQ ID NOs. 1 or 3, or set forth in OTTMUSG00000028740 or NCBI Reference number NM_001565.1, or a fragment thereof, for example, a cDNA fragment lacking the 3′ UTR.

Substantially Identical Sequences

By “substantially identical” is meant an amino acid or nucleotide sequence that differs from a reference sequence, such as a CXCL10, CXCL12 or CXCL13 sequence, only by one or more conservative substitutions, or by one or more non-conservative substitutions, deletions, or insertions located at positions of the sequence that do not destroy the biological function of the amino acid or nucleic acid molecule. Such a sequence can be any value from about 45% to about 99%, or more generally at least 45%, 48%, 50%, 52%, 55%, 57% or 60%, or at least 63%, 65%, 68%, 70%, 75%, 77%, 80%, 85%, 90%, or 95%, or as much as 96%, 97%, 98%, or 99% identical when optimally aligned at the amino acid or nucleotide level to the sequence used for comparison using, for example, the Align Program (Myers and Miller, CABIOS, 1989, 4:11-17) or FASTA. For polypeptides, the length of comparison sequences may be at least 10, 15, 20, 25, or 30 amino acids. In alternate embodiments, the length of comparison sequences may be at least 35, 40, or 50 amino acids, or over 60, 80, or 100 amino acids. For nucleic acid molecules, the length of comparison sequences may be at least 15, 20, 25, 30, 40, or 50 nucleotides. In alternate embodiments, the length of comparison sequences may be at least 60, 70, 80, or 90 nucleotides, or over 100, 200, or 500 nucleotides. Sequence identity can be readily measured using publicly available sequence analysis software (e.g., Sequence Analysis Software Package of the Genetics Computer Group, University of Wisconsin Biotechnology Center, 1710 University Avenue, Madison, Wis. 53705, or BLAST software available from the National Library of Medicine, or as described herein). Examples of useful software include the programs Pile-up and PrettyBox. Such software matches similar sequences by assigning degrees of homology to various substitutions, deletions, substitutions, and other modifications.

Alternatively, or additionally, two nucleic acid sequences may be “substantially identical” if they hybridize under high stringency conditions. In some embodiments, high stringency conditions are, for example, conditions that allow hybridization comparable with the hybridization that occurs using a DNA probe of at least 500 nucleotides in length, in a buffer containing 0.5 M NaHPO₄, pH 7.2, 7% SDS, 1 mM EDTA, and 1% BSA (fraction V), at a temperature of 65° C., or a buffer containing 48% formamide, 4.8×SSC, 0.2 M Tris-Cl, pH 7.6, 1× Denhardt's solution, 10% dextran sulfate, and 0.1% SDS, at a temperature of 42° C. (These are typical conditions for high stringency northern or Southern hybridizations.) Hybridizations may be carried out over a period of about 20 to 30 minutes, or about 2 to 6 hours, or about 10 to 15 hours, or over 24 hours or more. High stringency hybridization is also relied upon for the success of numerous techniques routinely performed by molecular biologists, such as high stringency PCR, DNA sequencing, single strand conformational polymorphism analysis, and in situ hybridization. In contrast to northern and Southern hybridizations, these techniques are usually performed with relatively short probes (e.g., usually about 16 nucleotides or longer for PCR or sequencing and about 40 nucleotides or longer for in situ hybridization). The high stringency conditions used in these techniques are well known to those skilled in the art of molecular biology, and examples of them can be found, for example, in Ausubel et al. (24).

Substantially identical sequences may, for example, be sequences that are substantially identical to the mouse or human CXCL10, CXCL12 or CXCL13 sequences described herein, or to homologous sequences found in in any mammalian species.

Oncolytic Viruses

Oncolytic viruses (OVs) are viruses that selectively replicate in cancer cells. As such, OVs may be capable of inducing the death of a cancer cell without having a significant effect on a non-cancer cell.

As used herein, an “oncolytic RNA virus” refers to an oncolytic virus that has ribonucleic acid (RNA) as its genetic material and induces inflammation by, for example, stimulating interferon production. In some embodiments, an oncolytic RNA virus does not persist in a tumour or cancer cell for a significant length of time i.e., is present transiently. For example, in some embodiments, an oncolytic RNA virus may be present in a tumour or cancer cell at levels that are 3 to 5 orders of magnitude less than the amount of inoculum at about 24 hours to about 72 hours following the last inoculum. In some embodiments, an oncolytic RNA virus may be present in a tumour or cancer cell at levels that are 1, 2, 3, 4, or 5 orders of magnitude less than the amount of inoculum at about 24 hours to about 72 hours following the last inoculum. In some embodiments, an oncolytic RNA virus may be present in a tumour or cancer cell at levels that are greater than 5 orders of magnitude less than the amount of inoculum at about 24 hours to about 72 hours following the last inoculum. It is to be understood that trace amounts (for example, less than 10% compared to the amount present 1 day after infection) of an oncolytic RNA virus may be present in a tumour or cancer cell 7 days after the last inoculum. In some embodiments, the oncolytic RNA virus may be completely cleared i.e., undetectable using standard detection techniques, from a tumour or cancer cell after about 14 days after the last inoculum.

Oncolytic RNA viruses include, without limitation, vesicular stomatitis virus (VSV), Maraba Virus, Reovirus, Measles virus, Poliovirus, or Newcastle Disease Virus.

In some embodiments, the oncolytic RNA virus is attenuated i.e., not pathogenic or capable of causing illness, but retaining its ability to infect cancer cells and stimulate an immune response.

In some embodiments, a VSV may include, without limitation, a VSV Indiana strain.

In some embodiments, a VSV may include, without limitation, a VSV including a mutation in the M protein.

In some embodiments, a VSV may include, without limitation, a VSV including a delta-51 mutation in the M protein, as described for example, in Stojdl, D F et al. (21). In some embodiments, a VSV may include, without limitation, a VSV having the sequence set forth in NCBI Reference Sequence: NC_001560.1 and further including a deletion of methionine 51 in the M protein.

In some embodiments, a Maraba Virus may include, without limitation, a Maraba Virus having the sequence set forth in NCBI Reference Sequence: NC_025255.1. In some embodiments, a Maraba Virus may include, without limitation, a Maraba Virus with L123W and Q242R mutations in the M and G proteins respectively, in the sequence set forth in NCBI Reference Sequence: NC_025255.1 (2).

In some embodiments, an oncolytic virus includes an oncolytic DNA virus. As used herein, an “oncolytic DNA virus” refers to an oncolytic virus that has deoxyribonucleic acid (DNA) as its genetic material. In some embodiments, an oncolytic DNA virus may replicate more slowly than an oncolytic RNA virus. In some embodiments, an oncolytic DNA virus may persist in tumours for longer periods of time than an oncolytic RNA virus. In some embodiments, an oncolytic DNA virus may not be a potent stimulator of type I interferons.

Oncolytic DNA viruses include, without limitation, Vaccinia Virus (VV), Herpes Simplex Virus (HSV), or Adenovirus.

In some embodiments, a VV may include, without limitation, a Vaccinia Virus Western Reserve strain (GenBank: AY243312.1), a Vaccinia Virus Acambis 2000 (GenBank: AY313847.1). In some embodiments, a VV may include, without limitation, a Vaccinia Virus having an attenuating mutation in the Thymidine Kinase (TK) locus, due to insertion of a heterologous sequence in that locus.

By “recombinant oncolytic virus,” as used herein, is meant an oncolytic RNA virus or an oncolytic DNA virus that expresses a heterologous B cell attractant polypeptide or a heterologous T cell attractant polypeptide.

By “recombinant,” as used herein, is meant the modification of a nucleic acid or amino acid sequence, resulting in a product that is not found in nature. When made in reference to a nucleic acid construct, the term refers to a molecule that is comprised of nucleic acid sequences that are joined together or produced by means of molecular biological techniques. The term “recombinant” when made in reference to a protein or a polypeptide refers to a protein or polypeptide molecule that is expressed using a recombinant nucleic acid construct created by means of molecular biological techniques. Recombinant nucleic acid constructs may include a nucleotide sequence which is ligated to, or is manipulated to become ligated to, a nucleic acid sequence to which it is not ligated in nature, or to which it is ligated at a different location in nature. Referring to a nucleic acid construct as ‘recombinant’ therefore indicates that the nucleic acid molecule has been manipulated using genetic engineering, i.e. by human intervention. Recombinant nucleic acid constructs may for example be introduced into a host cell by any suitable means described herein or known in the art. Such recombinant nucleic acid constructs may include sequences derived from the same host cell species or from different host cell species, which have been isolated and reintroduced into cells of the host species. Recombinant nucleic acid construct sequences may become integrated (“stably incorporated”) into a host cell genome, for example the genome of an oncolytic virus, either as a result of the original transformation of the host cells, or as the result of subsequent recombination and/or repair events.

By “heterologous” is meant a nucleic acid or polypeptide molecule that has been manipulated by human intervention so that it is located in a place other than the place in which it is naturally found. For example, a nucleic acid sequence from one species may be introduced into the genome of another species, or a nucleic acid sequence from one genomic locus may be moved to another genomic locus in the same species. A heterologous protein includes, for example, a protein expressed from a heterologous coding sequence or a protein expressed from a recombinant gene in a cell that would not naturally express the protein.

The term “recombinant,” when used in connection with an oncolytic virus, indicates that the oncolytic virus has been modified by the introduction of a heterologous nucleic acid sequence, such that the resulting recombinant oncolytic virus expresses a protein or polypeptide that is not normally expressed by the oncolytic virus, whether wild-type or attenuated. In some embodiments, a recombinant oncolytic virus may be engineered to express more than one heterologous nucleic acid sequence.

A recombinant VSV can be generated, for example, by inserting a heterologous nucleic acid sequence between the G and L proteins in the VSV genome, or between any two adjacent VSV genes. In some embodiments, the VSV may have a mutation in the M protein, or other mutations in VSV proteins that may confer tumour-selectivity. In some embodiments, the mutation at the M protein may be a deletion as described for example at position methionine 51.

An attenuated recombinant VV can be generated, for example, by inserting a heterologous nucleic acid sequence within the thymidine kinase locus, or vaccinia growth factor (VGF) locus, or any other locus where disruption of the gene confers tumour-selectivity.

In some embodiments, the heterologous nucleic acid sequence may include the 5′ UTR of the cDNA, the complete coding sequence, and the stop codon. In some embodiments, the heterologous nucleic acid sequence may omit the 3′ UTR, for example, if such an omission improves expression of the heterologous nucleic acid sequence. In some embodiments, a further heterologous 3′ UTR sequence may be introduced into the heterologous nucleic acid sequence cDNA to improve translation of the mRNA. In some embodiments, a further heterologous 3′ UTR sequence may be a synthetic sequence, as for example described by Levitt, N et al. (9).

When the oncolytic virus is a VV, the heterologous nucleic acid sequence may be placed under the control of the VV synthetic early/late promoter with the sequence:

(SEQ ID NO: 18)
AAAAATTGAAATTTTATTTTTTTTTTTTGGAATATAAATA.

Accordingly, in some embodiments, a recombinant oncolytic virus in accordance with the present disclosure refers to an oncolytic RNA or DNA virus that has been modified to express a B cell attractant polypeptide and includes, without limitation, a VSV-CXCL12, VV-CXCL12, VSV-CXCL13, or VV-CXCL13.

In some embodiments, a recombinant oncolytic virus in accordance with the present disclosure refers to an oncolytic RNA or DNA virus that has been modified to express a T cell attractant polypeptide and includes, without limitation, a VSV-CXCL10 or VV-CXCL10 virus as described herein.

It is to be understood that, while a recombinant oncolytic virus, expressing a heterologous polypeptide, may be undetectable after a certain period of time after inoculation at a particular location, the heterologous polypeptide may continue to be expressed by, for example, immune cells recruited by the recombinant oncolytic virus to that location and may therefore be detected.

Cancers

A recombinant oncolytic virus in accordance with the present disclosure may be used to recruit B cells and/or T cells to a solid cancer, tumour or neoplasm. By a “cancer,” “tumour” or “neoplasm” is meant any unwanted growth of cells serving no physiological function. In general, a cell of a neoplasm has been released from its normal cell division control, i.e., a cell whose growth is not regulated by the ordinary biochemical and physical influences in the cellular environment. In most cases, a neoplastic cell proliferates to form a clone of cells which are either benign or malignant. Examples of cancers or neoplasms include, without limitation, transformed and immortalized cells, tumours, and carcinomas such as breast cell carcinomas and prostate carcinomas. The term cancer includes cell growths that are technically benign but which carry the risk of becoming malignant.

By “malignancy” is meant an abnormal growth of any cell type or tissue. The term malignancy includes cell growths that are technically benign but which carry the risk of becoming malignant. This term also includes any cancer, carcinoma, neoplasm, neoplasia, or tumor. Most cancers fall within three broad histological classifications: carcinomas, which are the predominant cancers and are cancers of epithelial cells or cells covering the external or internal surfaces of organs, glands, or other body structures (e.g., skin, uterus, lung, breast, prostate, stomach, bowel), and which tend to mestastasize; sarcomas, which are derived from connective or supportive tissue (e.g., bone, cartilage, tendons, ligaments, fat, muscle); and hematologic tumors, which are derived from bone marrow and lymphatic tissue. Carcinomas may be adenocarcinomas (which generally develop in organs or glands capable of secretion, such as breast, lung, colon, prostate or bladder) or may be squamous cell carcinomas (which originate in the squamous epithelium and generally develop in most areas of the body). Sarcomas may be osteosarcomas or osteogenic sarcomas (bone), chondrosarcomas (cartilage), leiomyosarcomas (smooth muscle), rhabdomyosarcomas (skeletal muscle), mesothelial sarcomas or mesotheliomas (membranous lining of body cavities), fibrosarcomas (fibrous tissue), angiosarcomas or hemangioendotheliomas (blood vessels), liposarcomas (adipose tissue), gliomas or astrocytomas (neurogenic connective tissue found in the brain), myxosarcomas (primitive embryonic connective tissue), or mesenchymous or mixed mesodermal tumors (mixed connective tissue types). In addition, mixed type cancers, such as adenosquamous carcinomas, mixed mesodermal tumors, carcinosarcomas, or teratocarcinomas also exist.

Cancers may also be named based on the organ in which they originate i.e., the “primary site,” for example, cancer of the breast, brain, lung, liver, skin, prostate, testicle, bladder, colon and rectum, cervix, uterus, etc. This naming persists even if the cancer metastasizes to another part of the body that is different from the primary site. Cancers named based on primary site may be correlated with histological classifications. For example, lung cancers are generally small cell lung cancers or non-small cell lung cancers, which may be squamous cell carcinoma, adenocarcinoma, or large cell carcinoma; skin cancers are generally basal cell cancers, squamous cell cancers, or melanomas. Lymphomas may arise in the lymph nodes associated with the head, neck and chest, as well as in the abdominal lymph nodes or in the axillary or inguinal lymph nodes. Identification and classification of types and stages of cancers may be performed by using for example information provided by the Surveillance, Epidemiology, and End Results (SEER) Program of the National Cancer Institute (http://seer.cancer.gov/publicdata/access.html), which is an authoritative source of information on cancer incidence and survival in the United States and is recognized around the world. The SEER Program currently collects and publishes cancer incidence and survival data from 14 population-based cancer registries and three supplemental registries covering approximately 26 percent of the US population. The program routinely collects data on patient demographics, primary tumor site, morphology, stage at diagnosis, first course of treatment, and follow-up for vital status, and is the only comprehensive source of population-based information in the United States that includes stage of cancer at the time of diagnosis and survival rates within each stage. Information on more than 3 million in situ and invasive cancer cases is included in the SEER database, and approximately 170,000 new cases are added each year within the SEER coverage areas. The incidence and survival data of the SEER Program may be used to access standard survival for a particular cancer site and stage. For example, to ensure an optimal comparison group, specific criteria may be selected from the database, including date of diagnosis and exact stage.

The following list provides some non-limiting examples of primary cancers and their common sites for secondary spread (metastases):

| Primary cancer | Common sites for metastases |
|---|---|
| breast | bone, lungs, skin, brain |
| lung | bone, brain |
| colon | liver, lungs, bone |
| kidney | lungs, bone |
| pancreas | liver, lungs, bone |
| melanoma | lungs |
| uterus | lungs, bones, ovaries |
| ovary | liver, lung |
| bladder | bone, lung |

In some embodiments, the present disclosure includes cancers that are benefited by the recruitment of B cells, such as breast cancer, colorectal cancer, lung cancer, melanoma, or ovarian cancer. In some embodiments, the present disclosure includes cancers that are benefited by the recruitment of T cells. Without being bound to any particular theory, exogenous CXCL10 produced from an oncolytic virus may be particularly useful in cancers where CXCL10 expression has been silenced by genetic or epigenetic means.

Pharmaceutical & Veterinary Compositions, Dosages, and Administration

Recombinant oncolytic viruses, as described herein, can be formulated with a carrier, such as a pharmaceutically acceptable carrier, in a form suitable for administration to a subject. In some embodiments, ex vivo techniques may be used. For example, in some embodiments, the carrier may be an ex vivo infected autologous tumour cell, as described by Lemay C G et al. (8).

As used herein, a subject may be a human, non-human primate, rat, mouse, cow, horse, pig, sheep, goat, dog, cat, etc. The subject may be a clinical patient, a clinical trial volunteer, an experimental animal, etc. The subject may be at risk for having a cancer or neoplasm, be diagnosed with a cancer or neoplasm, or be a control subject that is confirmed to not have a cancer or neoplasm. Diagnostic methods for a cancer or neoplasm and the clinical delineation of such diagnoses are known to those of ordinary skill in the art.

One or more recombinant oncolytic viruses expressing heterologous polypeptides, such as CXCL10, CXCL12 or CXCL13, may be administered to a subject. For example, a subject may be administered one or more recombinant oncolytic viruses, such as VSV or VV, each expressing one or more of CXCL10, CXCL12 or CXCL13.

In some embodiments, a recombinant oncolytic RNA virus, such as a VSV-CXCL12 or VSV-CXCL13 virus, as described herein, can be provided alone or in combination with other compounds (for example, nucleic acid molecules, small molecules, peptides, peptide analogues, or a recombinant oncolytic DNA virus). For example, a VSV-CXCL12 or VSV-CXCL13 virus can be provided in combination with a VV-CXCL12, a VV-CXCL13 virus, a VSV-CXCL10 virus and/or a VV-CXCL10 virus.

If desired, treatment with a recombinant oncolytic RNA virus according to the present disclosure may be combined with more traditional and existing therapies for a cancer or neoplasm. A recombinant oncolytic RNA virus according to the present disclosure may be provided chronically or intermittently. “Chronic” administration refers to administration of the agent(s) in a continuous mode as opposed to an acute mode, so as to maintain the initial therapeutic effect (activity) for an extended period of time. “Intermittent” administration is treatment that is not consecutively done without interruption, but rather is cyclic in nature.

Conventional pharmaceutical practice may be employed to provide suitable formulations or compositions to administer a recombinant oncolytic RNA virus, by for example injection or inhalation, to a subject suffering from or suspected of having a cancer or neoplasm. Any appropriate route of administration may be employed, for example, parenteral, intravenous, subcutaneous, intracranial, intraorbital, ophthalmic, intraventricular, intracapsular, intraspinal, intrathecal, intracisternal, intraperitoneal, intranasal, aerosol, topical, or administration. Therapeutic formulations may be in the form of liquid solutions or suspensions; for intranasal formulations, in the form of nasal drops, or aerosols.

Methods well known in the art for making formulations are found in, for example, “Remington's Pharmaceutical Sciences” (19^{th} edition), ed. A. Gennaro, 1995, Mack Publishing Company, Easton, Pa. Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops. For therapeutic compositions, the compounds are administered to an individual in an amount sufficient to stop or slow a cancer or neoplasm.

A “therapeutically effective amount” refers to an amount effective, at dosages and for periods of time necessary, to achieve the desired therapeutic result, such as to stop or slow a cancer or neoplasm. A therapeutically effective amount of a compound may vary according to factors such as the disease state, age, sex, and weight of the individual, and the ability of the compound to elicit a desired response in the individual. Dosage regimens may be adjusted to provide the optimum therapeutic response. A therapeutically effective amount is also one in which any toxic or detrimental effects of the compound are outweighed by the therapeutically beneficial effects. In some embodiments, a therapeutically effective amount may be 1e4, 1e5, 1e6, 1e7, 1e8, 1e9, 1e10, 1e11, 1e12, 1e13, 1e14, 1e15 or more plaque forming units (pfu) per kg subject of a recombinant oncolytic virus as described herein.

It is to be noted that dosage values may vary with the severity of the condition to be alleviated, or the particular recombinant oncolytic virus used. For any particular subject, specific dosage regimens may be adjusted over time according to the individual need and the professional judgement of the person administering or supervising the administration of the compositions. Dosage ranges set forth herein are exemplary only and do not limit the dosage ranges that may be selected by medical practitioners. The amount of active recombinant oncolytic virus(es) in the composition may vary according to factors such as the disease state, age, sex, and weight of the individual subject. Dosage regimens may be adjusted to provide the optimum therapeutic response. For example, a single bolus may be administered, several divided doses may be administered over time or the dose may be proportionally reduced or increased as indicated by the exigencies of the therapeutic situation. It may be advantageous to formulate parenteral compositions in dosage unit form for ease of administration and uniformity of dosage.

Methods of Use

Recombinant oncolytic viruses, as described herein, can be used to inhibit the growth of a tumour, promote the killing of a tumour cell, or recruit immune cells (such as T cells or B cells) to a tumour.

By “inhibit the growth of a tumour” is meant a decrease by any value between 10% and 90%, or of any value between 30% and 60%, or over 100%, or a decrease by 1-fold, 2-fold, 5-fold, 10-fold or more of the size of a tumour in the presence of a recombinant oncolytic virus, as described herein, when compared to a similar tumour in the absence of the recombinant oncolytic virus. It is to be understood that the inhibiting does not require full inhibition.

By “promote the killing of a tumour cell” is meant an increase by any value between 10% and 90%, or of any value between 30% and 60%, or over 100%, or an increase by 1-fold, 2-fold, 5-fold, 10-fold, 15-fold, 25-fold, 50-fold, 100-fold or more in the death of a tumour cells in the presence of a recombinant oncolytic virus, as described herein, when compared to a similar tumour in the absence of the recombinant oncolytic virus. It is to be understood that the killing does not require that all tumour cells be killed.

By “recruit immune cells (to a tumour” is meant is meant an increase by any value between 10% and 90%, or of any value between 30% and 60%, or over 100%, or an increase by 1-fold, 2-fold, 5-fold, 10-fold, 15-fold, 25-fold, 50-fold, 100-fold or more in the number of immune cells, such as T cells or B cells, in the presence of a recombinant oncolytic virus, as described herein, when compared to number of immune cells, such as T cells or B cells, in the absence of the recombinant oncolytic virus.

Any suitable assay, as described herein or known in the art, can be used. For example, Western blotting can be used to detect the production of the heterologous protein by infected cells; the movement of B cells can be determined by transwell migration assays and the specificity of migration assessed using specific monoclonal antibodies to block migration. The NOP mammary cancer animal model can be used to assess the capacity of the recombinant virus to enhance anti-tumor efficacy by injecting the mice with virus and monitoring tumor growth. Mice can be sacrificed at serial time points to compare the numbers and activation status of tumor-infiltrating B cells and to monitor the formation of lymphoid clusters using flow cytometry and/or multicolour immunohistochemistry.

The present invention will be further illustrated in the following examples.

### EXAMPLES

Generation of Recombinant Viruses

We engineered the chemokines CXCL10 and CXCL13 into attenuated strains of VSV and VV. In the VSV recombinant viruses, the chemokine was inserted in between the G and L proteins in the VSV genome. For the VV recombinant viruses, the chemokine was inserted at the thymidine kinase locus. For both viruses, the transgene included the 5′ UTR of the cDNA, the complete coding sequence, and the stop codon; the 3′ UTR was omitted. We used PCR cloning to insert the murine CXCL13 (mCXCL13) gene into VSV. We amplified this from cDNA prepared from mRNA extracted from mouse splenocytes. Primers for this amplification included restriction enzyme sites to allow for insertion into the VSV viral genome. Upon obtaining recombinant VSV clones, we confirmed that the insertion of mCXCL13 was successful by Sanger sequencing. Recombinant VSV-mCXCL13 virus was generated from the DNA construct. For VV, we also PCR amplified the DNA for mCXCL13 from the mouse splenocyte cDNA. In this case PCR primers were designed such that they would allow for the insertion of mCXCL13 into the VV viral genome. In addition, the left primer for VV was designed to contain a synthetic promoter to force high expression of mCXCL13 in target cells infected with the recombinant virus.

Cloning Chemokine Genes into Recombinant Oncolytic Virus Plasmids

More specifically, to isolate cDNA from activated mouse splenocytes, a freshly-harvested mouse spleen was mashed with the blunt end of a syringe plunger then filtered through a 100 μm screen. Splenocytes were pelleted and re-suspended in ACK lysis buffer. Following five minutes of incubation at room temperature, cells were washed then re-suspended in complete RPMI prior to filtration through a 40 μm strainer. Splenocytes were grown at a concentration of 1-2×10⁶ cells/mL in complete RPMI media. Concavalin A (Sigma-Aldrich) was used to stimulate the cells at a concentration of 1 μg/mL. Cells were incubated for 48 hr then pelleted by centrifugation and re-suspended in a solution of buffer RLT Plus (Qiagen) with 1% β-mercaptoethanol prior to homogenization. The RNeasy Plus Mini Kit (Qiagen) was used to extract RNA from the homogenate following the manufacturer's protocol. cDNA was prepared from RNA using qScript™ cDNA SuperMix (Quanta Biosciences) following the manufacturer's protocol.

The primers in Table 1 were used to amplify CXCL10 and CXCL13 cDNA lacking the 3′ UTR from total mouse cDNA. For VSV cloning, primers were designed to contain xhol and Nhel restriction sites. For Vaccinia Virus cloning, primers were designed to contain spel restriction sites. Additionally, the left Vaccinia Virus primers contained a synthetic Vaccinia Virus early/late promoter:

(SEQ ID NO: 18; 3)
AAAAATTGAAATTTTATTTTTTTTTTTTGGAATATAAATA

to drive high expression of the chemokine genes. All primers were purchased from Integrated DNA Technologies. PCR was performed using the high-fidelity Q5 polymerase (NEB).

TABLE 1

| | | | Restriction |
|---|---|---|---|
| Primer Name | Sequence (5′ to 3′) | Target | site |
| VSV_CXCL13_left | TAAGCACTCGAGGAGCTAAAGG | mCXCL13 | xhoI |
| | TTGAACTCCAC (SEQ ID NO: 19) | cDNA | |
| VSV_CXCL13_right | TGCTTAGCTAGCTCAGGCAGCT | mCXCL13 | NheI |
| | CTTCTCTTACT (SEQ ID NO: 20) | cDNA | |
| VSV_CXCL10_left | TAAGCACTCGAGGAGAAGCGCT | mCXCL10 | xhoI |
| | TCATCCACCG (SEQ ID NO: 21) | cDNA | |
| VSV_CXCL10_right | TGCTTAGCTAGCTTAAGGAGCC | mCXCL10 | NheI |
| | CTTTTAGACCT (SEQ ID NO: 22) | cDNA | |
| VV_CXCL13_left | GATCCAACTAGTAAAAATTGAAA | mCXCL13 | speI |
| | TTTTATTTTTTTTTTTTGGAATAT | cDNA | |
| | AAATAGAGCTAAAGGTTGAACT | | |
| | CCAC (SEQ ID NO: 23) | | |
| VV_CXCL13_right | TGCTTAACTAGTTCAGGCAGCT | mCXCL13 | speI |
| | CTTCTCTTACT (SEQ ID NO: 24) | cDNA | |
| VV_CXCL10_left | GATCCAACTAGTAAAAATTGAAA | mCXCL10 | speI |
| | TTTTATTTTTTTTTTTTGGAATAT | cDNA | |
| | AAATAGAGAAGCGCTTCATCCA | | |
| | CCG (SEQ ID NO: 25) | | |
| VV_CXCL10_right | TGCTTAACTAGTTTAAGGAGCC | mCXCL10 | speI |
| | CTTTTAGACCT (SEQ ID NO: 26) | cDNA | |
| VV_TK_Fwd | ATGAACGGCGGACATATTCAGT | VV | NA |
| | (SEQ ID NO: 27) | Thymidine | |
| | | Kinase | |
| VV_TK_Rev | GAGTCGATGTAACACTTTCTAC | VV | NA |
| | (SEQ ID NO: 28) | Thymidine | |
| | | Kinase | |

VSV constructs were cloned into the VSV-d51 plasmid (21) and Vaccinia Virus constructs were cloned into plasmid pSEM-1 plasmid (19) using standard cloning techniques. The VSV-d51 plasmid allows insertion of the transgene between the G and L genes, and the pSEM-1 plasmid allows insertion into the Thymidine Kinase (TK) locus. Once chemokine expression constructs had been cloned into their respective recombinant virus plasmids, the chemokine construct was sequenced (Genscript) to ensure there were no errors before proceeding to generate recombinant virus.

The chemokine constructs were confirmed to have at least the following sequences:

mCXCL10 cDNA lacking the 3′ UTR:
(SEQ ID NO: 1)
GAGAAGCGCTTCATCCACCGCTGAGAGACATCCCGAGCCAACCTTCCGG
AAGCCTCCCCATCAGCACCATGAACCCAAGTGCTGCCGTCATTTTCTGC
CTCATCCTGCTGGGTCTGAGTGGGACTCAAGGGATCCCTCTCGCAAGGA
CGGTCCGCTGCAACTGCATCCATATCGATGACGGGCCAGTGAGAATGAG
GGCCATAGGGAAGCTTGAAATCATCCCTGCGAGCCTATCCTGCCCACGT
GTTGAGATCATTGCCACGATGAAAAAGAATGATGAGCAGAGATGTCTGAA
TCCGGAATCTAAGACCATCAAGAATTTAATGAAAGCGTTTAGCCAAAAAA
GGTCTAAAAGGGCTCCTTAA
mCXCL13 cDNA lacking the 3′ UTR:
(SEQ ID NO: 5)
GAGCTAAAGGTTGAACTCCACCTCCAGGCAGAATGAGGCTCAGCACAGC
AACGCTGCTTCTCCTCCTGGCCAGCTGCCTCTCTCCAGGCCACGGTATT
CTGGAAGCCCATTACACAAACTTAAAATGTAGGTGTTCTGGAGTGATTTC
AACTGTTGTCGGTCTAAACATCATAGATCGGATTCAAGTTACGCCCCCTG
GGAATGGCTGCCCCAAAACTGAAGTTGTGATCTGGACCAAGATGAAGAA
AGTTATATGTGTGAATCCTCGTGCCAAATGGTTACAAAGATTATTAAGAC
ATGTCCAAAGCAAAAGTCTGTCTTCAACTCCCCAAGCTCCAGTGAGTAAG
AGAAGAGCTGCCTGA

Generation of Recombinant VSV

To generate recombinant VSV, we used an established recombinant virus rescue protocol (7). 5e5 Vero (ATCC CCL-81) cells/well were plated in a 6 well tissue culture plate in 2 ml of complete media (500 ml High Glucose (4500 mg/L) DMEM, 50 ml Heat Inactivated fetal bovine serum, 5 ml each of penicillin/streptomycin, 2 mM L-Glutamine and 1 mM Sodium Pyruvate (Thermo Fisher Scientific). 24 hours later when cells had formed a confluent monolayer, media was removed, and the cells were infected with a T7-expressing Vaccinia (VV-T7) at an MOI of 5 (5e6 pfu) in a volume of 100 ul of serum-free High Glucose DMEM per well. 2 hours after infection, a transfection mix contain the following components was prepared: 1 ug/well of VSV-N plasmid, 1.25 ug/well of VSV-P plasmid, 0.25 ug/well of VSV-L plasmid, and 4 ug/well of recombinant VSV genome plasmid. The final volume per transfection was made up to 250 ul in Opti-MEM reduced serum media (Thermo Fisher Scientific).

In a separate tube, 5 ul of lipofectamine 2000 (Thermo Fisher Scientific) was added to 250 ul Opti-MEM. The plasmid and lipofectamine solutions were mixed together and incubated at room temperature for 10-20 minutes.

Supernatants from wells infected with VV-T7 were removed by aspirating the inoculum, and 500 ul/well of the transfection mixture was added dropwise directly onto the cells. One VV-T7 infected well was left un-transfected as a negative control. Plates were incubated in the transfection solution for 4-5 hours at 37 C. Following this incubation, the transfection solution was aspirated and replaced with 2ml/well of complete media. Plates were incubated for 2 days.

Following the 2 day incubation, cultures were harvested and centrifuged at 1600 RPM for 10 minutes to pellet cell debris. The supernatant was then passed through a 0.2 um filter to remove any Vaccinia Virus contaminants.

1 ml of this clarified supernatant was plated onto a fresh, confluent layer of 6e5 Vero cells/well of a 6-well tissue culture plate and incubated at 37 C for 24-48 h. Successful recombinant virus rescue was indicated by cell death within 24-48h.

Successfully rescued wells were pooled together and frozen at −80 C. These stocks were used to generate subsequent virus stocks used in experiments.

Confirmation of Chemokine Expression in Recombinant VSV

6e5 Vero cells in 2 mL of complete media per well were plated in a 6-well tissue culture plate. The following day, the media was removed and cells were infected with supernatants containing the rescued recombinant VSV-CXCL13. The infections were performed using various amounts of supernatant made up to 500 μL total in serum-free High Glucose DMEM (Thermo Fisher Scientific). Virus was added to Vero cells and then the cells were incubated at 37 C for 1 hour, gently rocking the plates every 15 minutes. Wells were then topped up with 1.5 mL of 2% FBS High Glucose DMEM media and incubated at 37 C. The following day, a cell scraper (Sarstedt) was used to harvest the cells which were then centrifuged at 1500 rpm for 10 minutes. The supernatant was discarded and the pellet was re-suspended in 350 μL of RLT Lysis buffer (Qiagen) containing β-Mercaptoethanol (Thermo Fisher Scientific). Next, RNA was extracted using the RNEasy Kit (Qiagen). RNA was converted to cDNA using the qScript cDNA Kit (Quanta Biosciences). Next, PCR was used to screen for expression of the chemokine genes from the VSV genome. PCR was carried out using the cloning primers used to generate recombinant viruses and Taq polymerase (Thermo Fisher Scientific). The PCR cycling conditions were: an initial denaturation at 95° C. for 30 seconds followed by 35 cycles consisting of 30 seconds at 95° C., 30 seconds at 54.5° C. and 35 seconds at 72° C. were done, with a 2 minute long final extension step at 72° C. PCR products were visualized on a 2% agarose gel. For both CXCL10 and CXCL13 recombinant VSV, we were able to detect a band of the expected size indicating the transgene is transcribed.

Generation of Recombinant Vaccinia Virus

To generate recombinant Vaccinia Virus strains, we adapted the method previously described by Rintoul et al. (19). Briefly, 9e5 U-2 OS cells (ATCC HTB-96) were plated in 2 ml of complete media (500 ml High Glucose (4500 mg/L) DMEM, 50 ml Heat Inactivated fetal bovine serum, 5 ml each of penicillin/streptomycin, 2 mM L-Glutamine and 1 mM Sodium Pyruvate) (Thermo Fisher Scientific) in 6 well tissue culture plates and incubated at 37 C.

The following day, the helper virus (a wild type Vaccinia Virus Western Reserve Strain) was diluted in serum-free media. Media was aspirated from the wells, and cells were infected at an MOI of 3-5 in a volume of 300-500 ul per well and incubated at 37 C for 1 hour, gently rocking the plate every 15 minutes.

While cells were being infected, 10 ul of lipofectamine 2000 (Thermo Fisher Scientific) was added to 250 ul of reduced-serum Opti-MEM media (Thermo Fisher Scientific). 4 ug of recombinant plasmid DNA was added to an equal volume (250 ul) of Opti-MEM and then combined with the lipofectamine mixture. The lipofectamine/plasmid mixture was incubated at room temperature for 10-20 minutes.

After the 1 hour virus infection, media was aspirated from the wells, and the transfection mixture was added dropwise to the wells. As a negative control, one well was infected with helper virus, but left un-transfected. Plates were incubated at 37 C for 3-4 hours. Following this incubation, the transfection mixture was aspirated and replaced with 2 ml/well of High Glucose DMEM media. Plates were then incubated for 24-48 h at 37 C.

Following this incubation, the contents of the wells were harvested using a cell scraper (Sarstedt) and spun at 3000 RPM for 10 minutes. The supernatant was discarded and the pellet was re-suspended in 200 ul (per original well in the 6-well plate) of 1 mM Tris, pH9. This virus mixture was transferred to a cryovial and subjected to 3 freeze-thaw cycles (−80 C and 37 C).

Next, in a 6 well plate containing confluent U2-OS cells, media was removed and between 5 and 20 ul of freeze-thawed virus was mixed with serum-free media to bring the total volume to 500 ul and plated for 1 hour. After the 1 hour incubation, the virus mixture was removed and 2 ml/well of GPT selection complete High Glucose DMEM media (containing 250 μg/mL xanthine, 15 μg/mL hypoxanthine (Sigma), and 25 μg/mL mycophenolic (MPA, Merck Millipore)) was added to each well. Plates were incubated at 37° C. for between 24-96 hours.

Plates were checked daily under a fluorescent microscope for the presence of the YFP selectable marker. When YFP+ve colonies reached an appreciable size they were picked directly under the fluorescent microscope into 100-150 ul of 1 mM Tris, pH9. Virus picked by this method was freeze thawed 3 times (−80 C to 37 C) and then plated on fresh U2-OS cells in a 6 well plate in the presence of GPT selection as indicated above.

In total, the virus went through 4-5 rounds of GPT selection/plaque picking. The final crude stock of virus was stored at −80 C and used to generate subsequent virus stocks.

Confirmation of Recombinant Vaccinia Virus Purity

To confirm that there were no wild type virus contaminants in our recombinant virus preparations we first infected U2-OS cells with the recombinant virus and then extracted DNA from the resultant cell/virus mixture as described by Meyer et al. (12). We used Platinum® Taq DNA Polymerase High Fidelity (Invitrogen) in combination with primers that anneal to the Vaccinia Virus Thymidine Kinase (TK) locus to screen for wild type contaminants. In wild type virus, the TK primer set amplifies a band of ˜500 bp. In the recombinant virus due to insertion into the TK locus, the TK primer set amplifies a region of ˜5 kb. Therefore the absence of the ˜500 bp band (but presence of the ˜5 kb band) indicates purity of the recombinant stock. PCR cycling conditions were as follows: one cycle of 94 C for two minutes; 40 cycles of 94 C for 30 seconds, 58 C for 30 seconds, 68 C for 5.5 minutes; one cycle of 68 C for 10 minutes; hold at 4 C. PCR products were visualized on a 0.8% agarose gel. Both CXCL10 and CXCL13 recombinant Vaccinia Virus stocks were found to be pure.

Production of Virus

VSV

Confluent monolayers of Vero cells were grown on 150 mm culture dishes. Each dish was infected with VSV at an MOI of roughly 0.02, with the virus diluted to 5 mL in serum-free media. After one hour of incubation with plate rocking every 15 minutes, 20 mL of 2% serum-containing media was added to each plate and incubated for 24 hours. Supernatants from infected cultures were harvested and centrifuged at 1400 rpm for 10 minutes and then filtered through a 0.2 μm filter (Thermo Fisher Scientific). The filtered supernatant was then centrifuged at 16000 rpm at 4 C for 90 minutes using the Avanti J-20 XP centrifuge with the JA-25.5 rotor (Beckman Coulter). After centrifugation, the supernatant was discarded and the viral pellets were pooled and re-suspended in 1 mL PBS per 10 plates. The virus was then aliquoted and stored at −80 C. Virus was titered using a standard plaque assay on Vero cells.

Vaccinia Virus

Confluent monolayers of U2-OS cells were grown on 150 mm culture dishes. Each dish was infected with Vaccinia Virus at 2e6 pfu per plate, diluted to 5 mL in serum-free media. After one hour of incubation with plate rocking every 15 minutes, 20 mL of 2% serum-containing media was added to each plate and incubated for ˜72 h hours. Infected cells were scraped and spun at 3000 rpm for 10 minutes. The pellet was re-suspended in 1 mM Tris-HCl pH 9 (4 mL per plate) and freeze-thawed three times. Tubes were spun at 3000 rpm for 10 minutes to remove cell debris. Cleared supernatant was overlaid onto 10 mL 36% sucrose solution (20 mL cleared supernatant per tube). Tubes were then spun at 11,500 rpm for 1.5 hours in the Avanti J-20 XP centrifuge using JS-13.1 swinging bucket rotor (Beckman Coulter, Pasadena, Calif.). Supernatant was poured off and excess sucrose was removed with a pipette. Pellets were resuspended in 1 mM Tris-HCl pH 9 (1 ml per 10 plates). Virus was aliquoted and stored at −80 C. Virus was titered using a standard plaque assay on U2-OS cells.

Confirmation of Chemokine Protein Production by Recombinant Viruses

To verify that the recombinant viruses produced chemokine protein upon infecting tumor cells, 3e4 NOP23 mouse mammary tumour cells (22) were plated in a 96-well plate and incubated for 24 hr. NOP23 tumour cells were then infected at MOI=1 with recombinant or parental (GFP expressing) viruses then incubated for 48 hrs. Media was collected, centrifuged, and chemokine in the culture supernatant was quantified by ELISA using the Mouse CXCL10/IP-10/CRG-2 or CXCL13/BLC/BCA-1 Quantikine ELISA Kits (R&D Systems,) following the manufacturer's protocol. Well colour intensity was analysed using a VersaMax Microplate Reader (Molecular Devices).

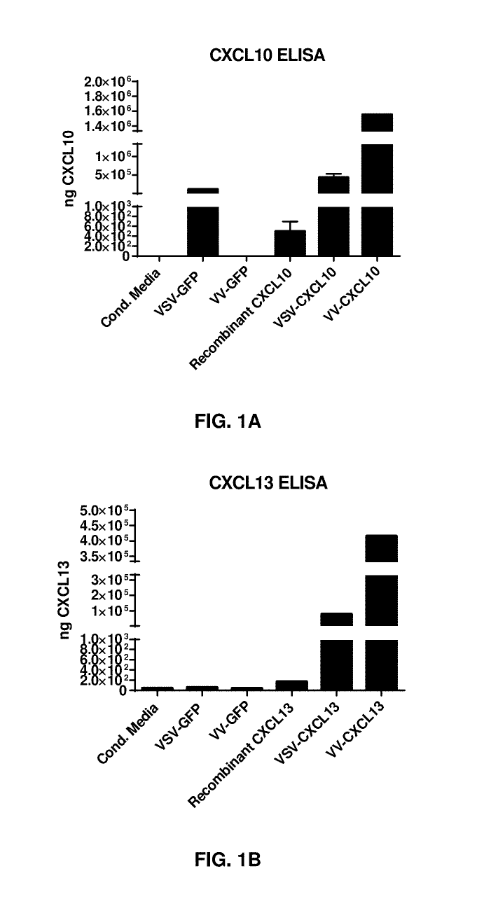

The ELISA revealed that parental VSV stimulated CXCL10 production in tumour cells, and this expression could be further increased by a CXCL10 transgene. In contrast, VV-GFP could not stimulate CXCL10 production in tumour cells, however the recombinant VV-CXCL10 stimulated robust production of CXCL10 (FIG. 1A).

Neither VSV-GFP nor VV-GFP infection induced CXCL13 expression from tumour cells. In contrast, both VSV-CXCL13 and VV-CXCL13 induced robust expression of CXCL13 in tumour cells (FIG. 1B).

In Vivo Assessment of VSV-CXCL13 Efficacy

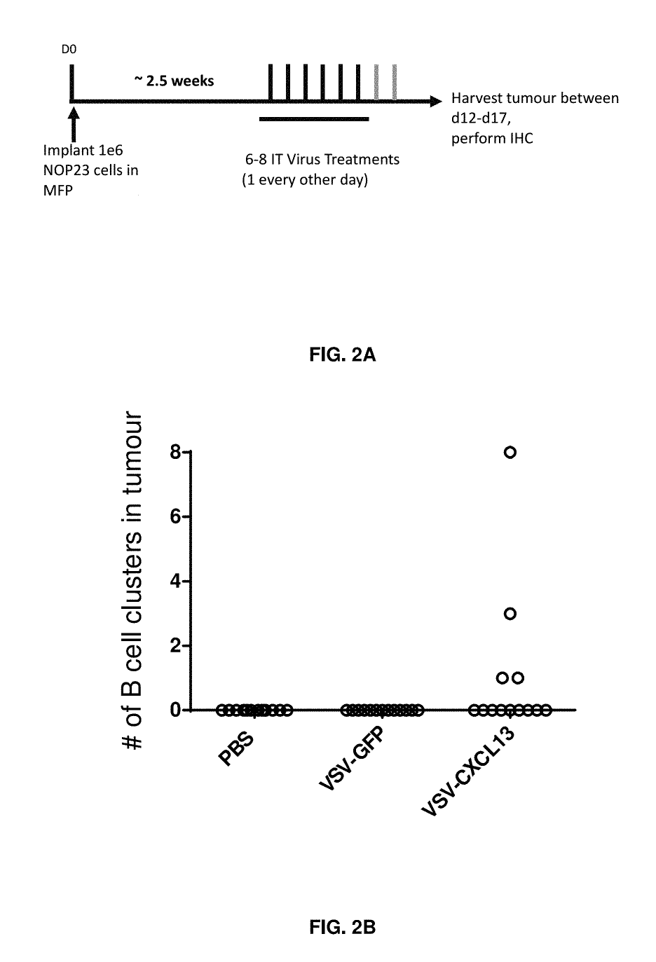

A mouse model of mammary cancer (22) was used to determine the therapeutic efficacy of VSV-CXCL13, and its ability to recruit B cells. The experimental approach used is shown in the schematic diagram in FIG. 2A.

More specifically, 1 e6 NOP23 mammary tumour cells were implanted into the mammary fat pad in a volume of 100 ul PBS. Roughly 3 weeks later when tumours had a reached size of ˜30-50 mm², animals received 6-8 intratumoural (one every other day) injections of PBS, or 5e8 pfu of VSVd51-GFP, or VSV-d51-CXCL13. Tumour size was monitored using digital calipers. Some cohorts of mice were euthanized 14 days after the 1^{st} virus/PBS treatment and their tumours were harvested into formalin to assess T and B cell infiltrates by immunohistochemistry. Tumour slides were stained with haematoxylin, anti-mouse CD3 with a brown 3,3′-Diaminobenzidine (DAB) chromogen, and an anti-mouse Pax5 antibody with Fast Red chromogen.

Immune cells and lymphoid clusters (large aggregates of CD3+ T cells and Pax5+ B cells) were counted in whole tumour sections (FIG. 2B). Tumours from PBS treated mice lacked immune cells, while VSV-GFP treated mice had a high density of T cells, but contained few B cells. Mice treated with VSV-CXCL13 contained T cell infiltrates and a subset of mice also contained B cell infiltrates. We did not detect any lymphoid clusters in mock (PBS) or VSV-GFP treated animals. In contrast, VSV-CXCL13 treatment induced lymphoid clusters in a subset of animals, and in some cases individual tumours contained multiple lymphoid clusters, indicating that treating tumours with VSV-CXCL13 can recruit B cells to tumours, and that these B cells often form lymphoid clusters. Data represent the combination of 3 independent experiments. N=12 for PBS; N=13 for VSV-GFP and VSV-CXCL13.

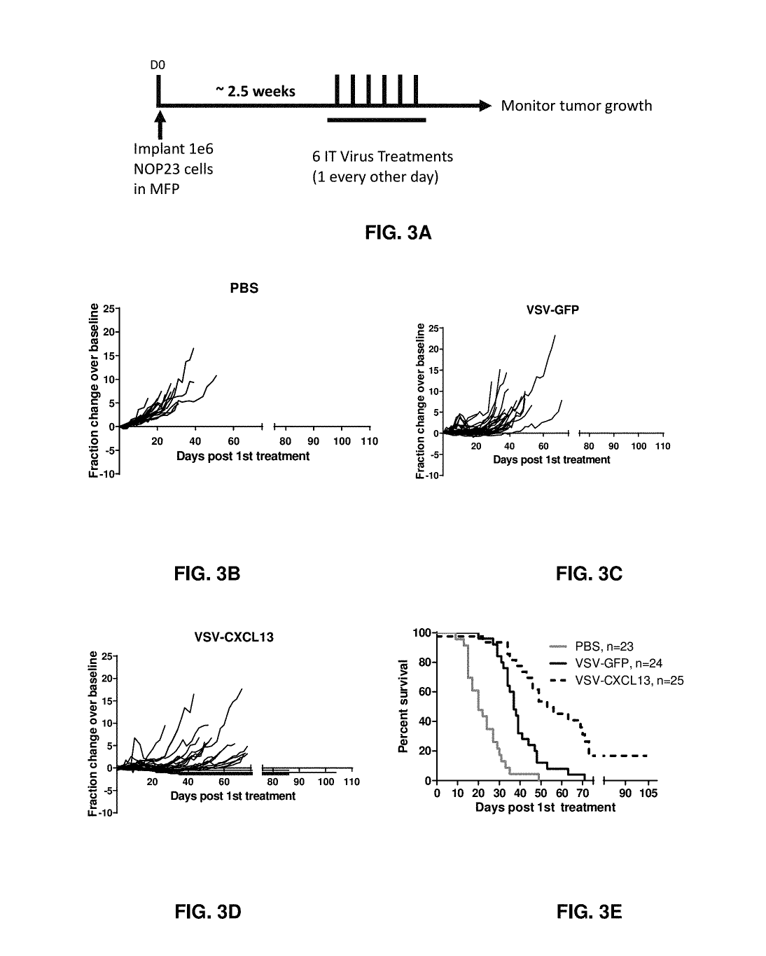

To determine the therapeutic efficacy of VSV-CXCL13, animals received 6 intratumoural (one every other day) injections of PBS, 5e8 pfu of VSVd51-GFP, or VSV-d51-CXCL13 (FIG. 3A). Tumour size was monitored using digital calipers. Animals were euthanized when they had reached endpoint, defined as tumours greater than or equal to 150 mm² in size. VSV-GFP treatment reduced the rate of tumour growth compared to mock (PBS) treated animals (FIGS. 3B,C). VSV-CXCL13 treatment was even more effective than VSV-GFP treatment, resulting in a statistically significant (p<0.0001) difference in tumour growth and survival (FIGS. 3D,E). In some cases, VSV-CXCL13 treated animals had complete, durable tumour regression (Figs. D,E). The data indicate that VSV-CXCL13 is therapeutically superior to parental (VSV-GFP) treatment. Data represent the combination of 3 independent experiments.

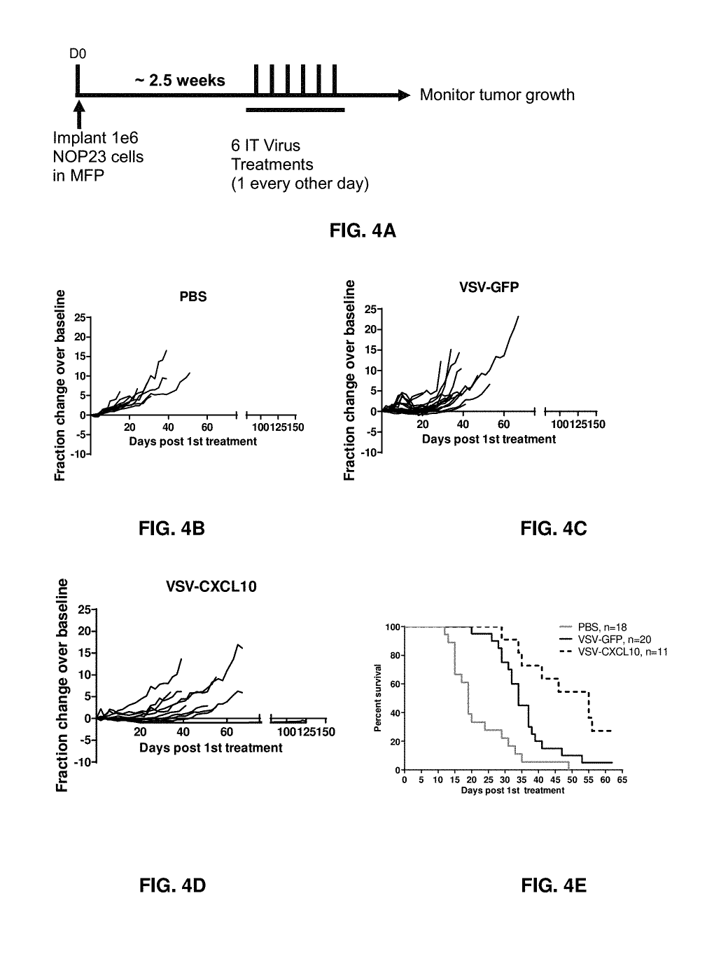

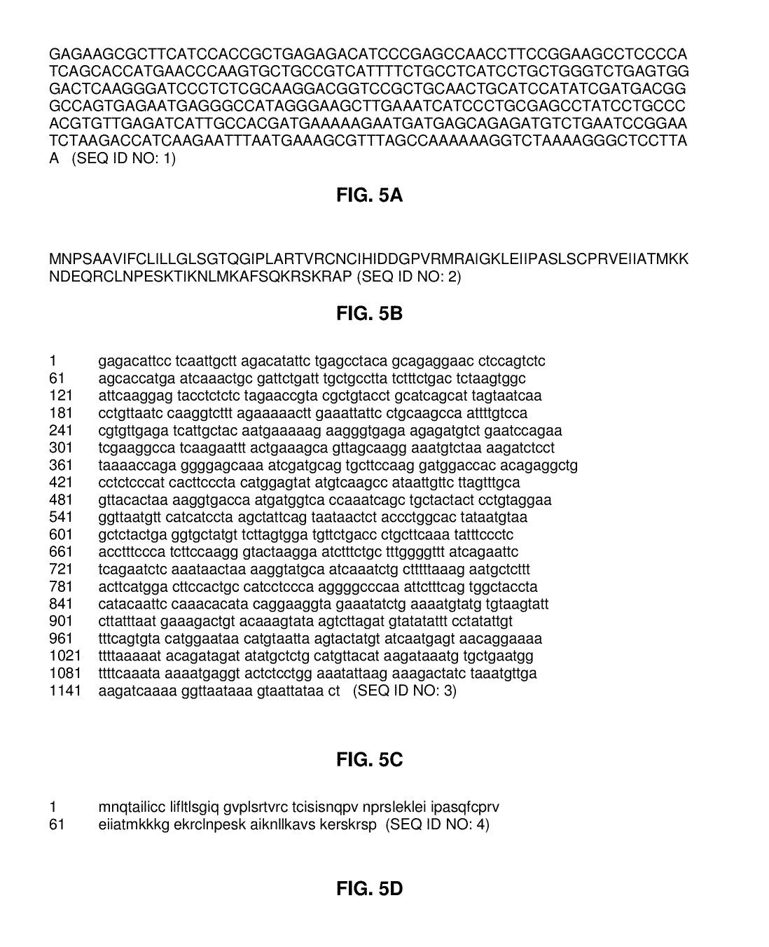

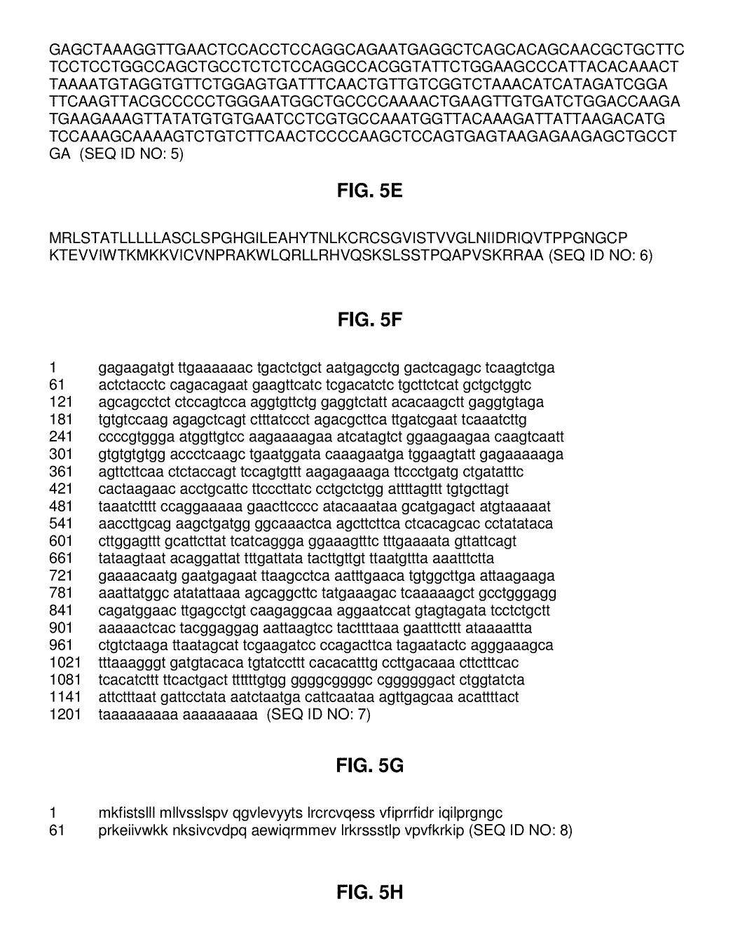

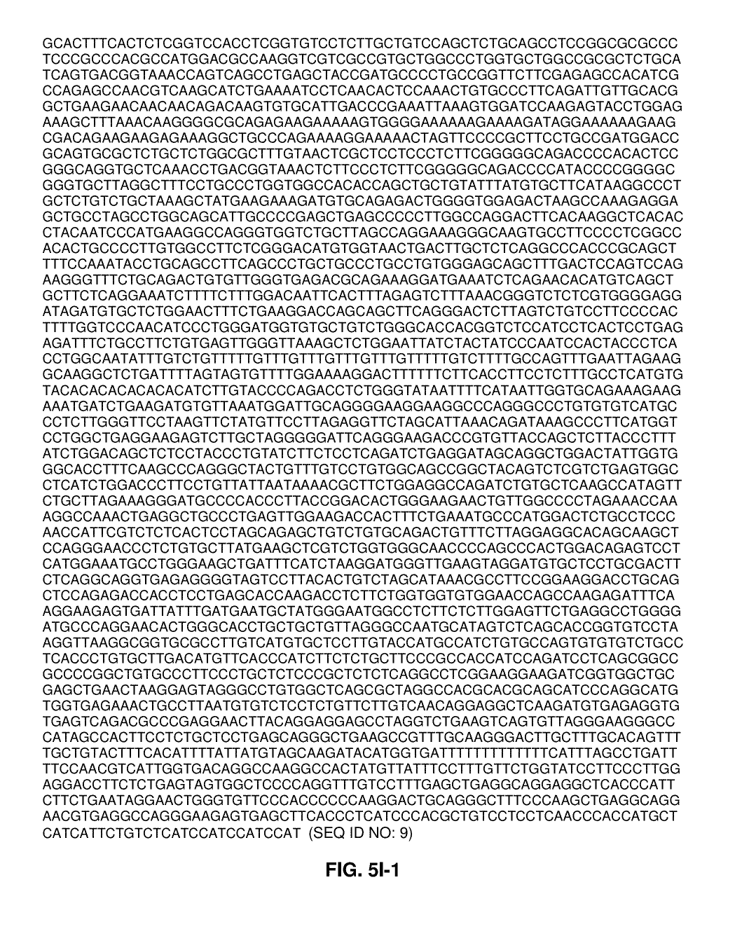

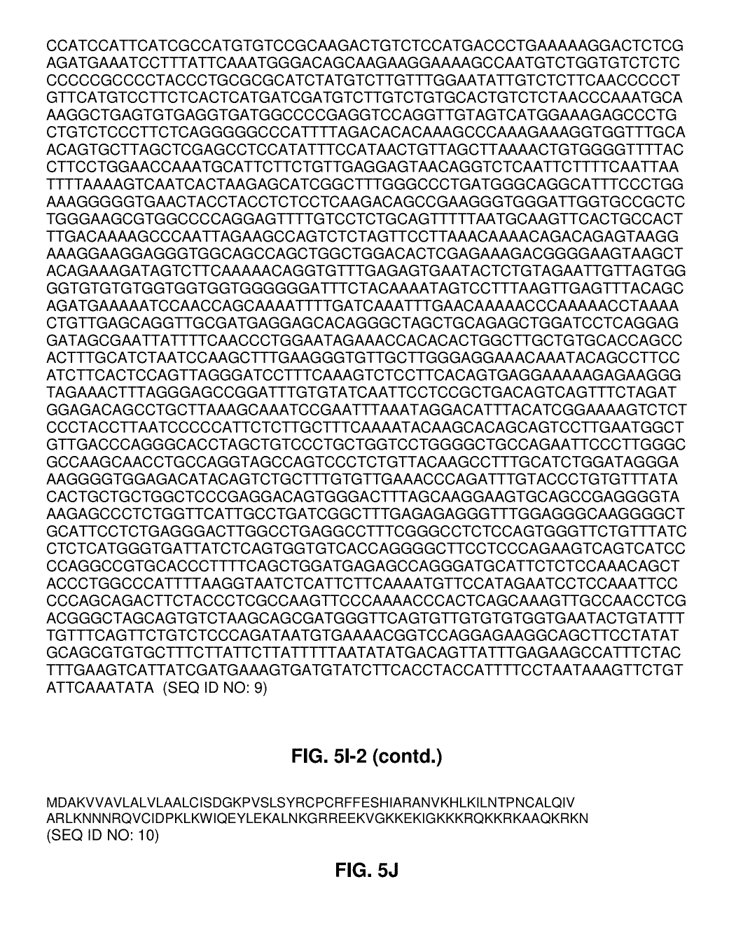

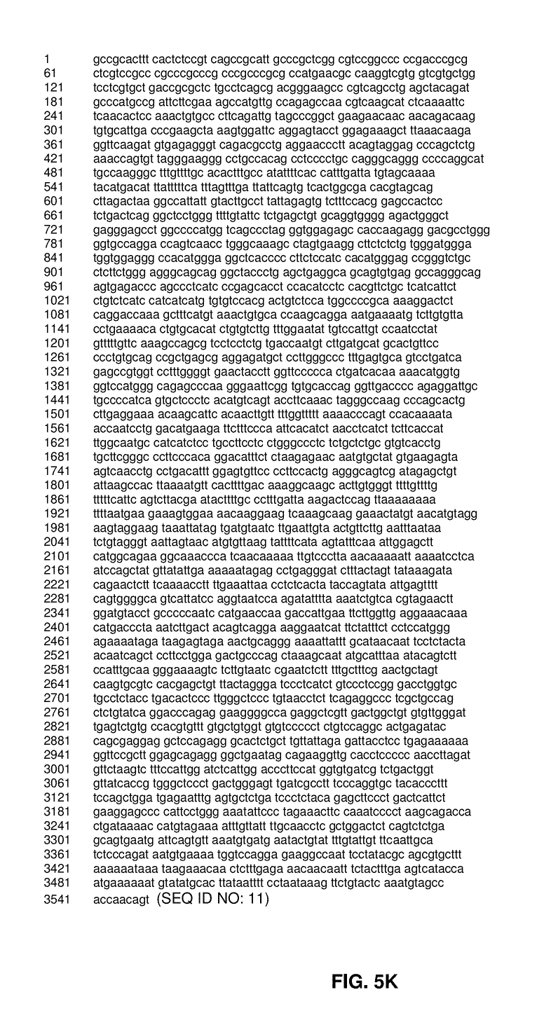

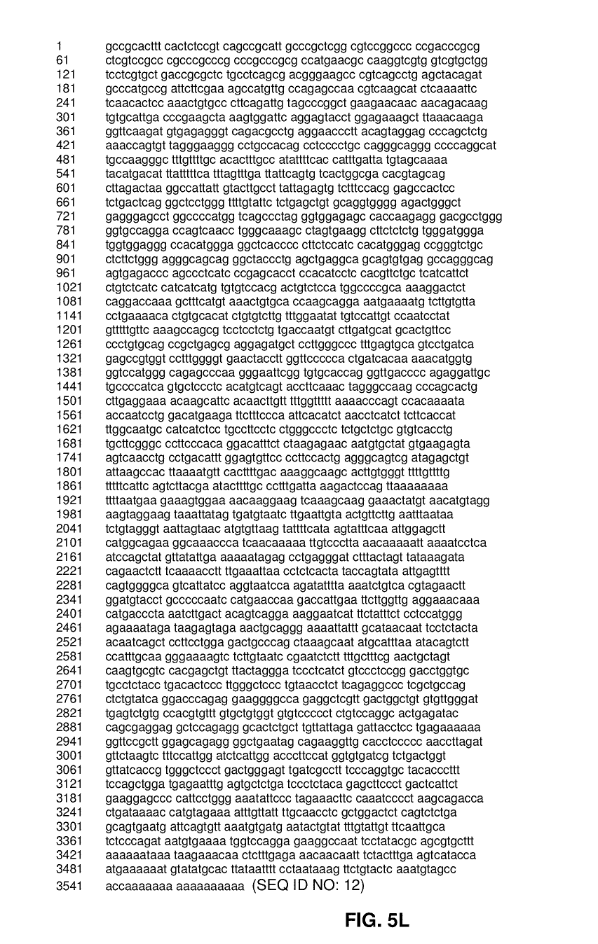

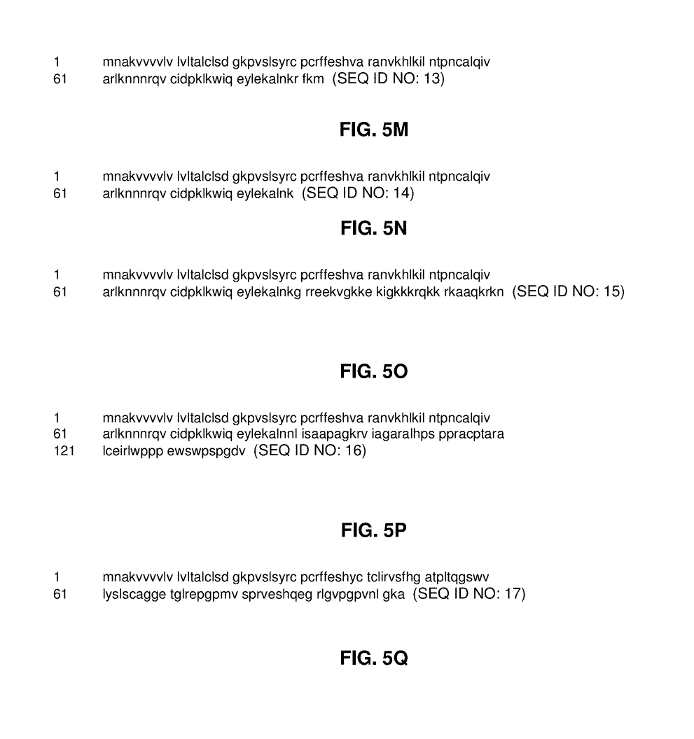

To determine the therapeutic efficacy of VSV-CXCL10, the NOP23 mouse model of mammary cancer (22) was used. As for VSV-CXCL13, 1 e6 NOP23 mammary tumour cells were implanted into the mammary fat pad in a volume of 100 ul PBS. Roughly 3 weeks later when tumours had a reached size of ˜30-50 mm², animals received 6 intratumoural (one every other day) injections of PBS, 5e8 pfu of VSVd51-GFP, or VSV-d51-CXCL10 (FIG. 4A). Tumour size was monitored using digital calipers. Animals were euthanized when they had reached endpoint, defined as tumours greater than or equal to 150 mm² in size. VSV-GFP treatment reduced the rate of tumour growth compared to mock (PBS) treated animals is (FIGS. 4B,C). VSV-CXCL10 treatment was even more effective that VSV-GFP treatment, resulting in a statistically significant (p=0.0073) difference in tumour growth and survival (FIGS. 4D,E). In some cases, VSV-CXCL10 treated animals had complete, durable tumour regression (FIGS. 4D,E). The data indicate that VSV-CXCL10 is therapeutically superior to parental (VSV-GFP) treatment. The data also suggest that, although the parental (VSV-GFP) virus induces CXCL10 expression, further increasing this expression with the CXCL10 transgene may have therapeutic benefit. Data represent the combination of 2 independent experiments.

### REFERENCES

1. Bindea G, Mlecnik B, Tosolini M, Kirilovsky A, Waldner M, et al. (2013) Spatiotemporal dynamics of intratumoral immune cells reveal the immune landscape in human cancer. Immunity 39: 782-795.
2. Brun J, McManus D, Lefebvre C, Hu K, Falls T, et al. (2010) Identification of genetically modified Maraba virus as an oncolytic rhabdovirus. Mol Ther 18: 1440-1449.
3. Chakrabarti S, Sisler J R, Moss B (1997) Compact, synthetic, vaccinia virus early/late promoter for protein expression. Biotechniques 23: 1094-1097.
4. Coppola D, Nebozhyn M, Khalil F, Dai H, Yeatman T, et al. (2011) Unique ectopic lymph node-like structures present in human primary colorectal carcinoma are identified by immune gene array profiling. Am J Pathol 179: 37-45.
5. Cripe T P, Ngo M C, Geller J I, Louis C U, Currier M A, et al. (2015) Phase 1 study of intratumoral Pexa-Vec (JX-594), an oncolytic and immunotherapeutic vaccinia virus, in pediatric cancer patients. Mol Ther 23: 602-608.
6. Goc J, Fridman W H, Sautes-Fridman C, Dieu-Nosjean M C (2013) Characteristics of tertiary lymphoid structures in primary cancers. Oncoimmunology 2: e26836.
7. Lawson N D, Stillman E A, Whitt M A, Rose J K (1995) Recombinant vesicular stomatitis viruses from DNA. Proc Natl Acad Sci USA 92: 4477-4481.
8. Lemay C G, Rintoul J L, Kus A, Paterson J M, Garcia V, et al. (2012) Harnessing oncolytic virus-mediated antitumor immunity in an infected cell vaccine. Mol Ther 20: 1791-1799.
9. Levitt N, Briggs D, Gil A, Proudfoot N J (1989) Definition of an efficient synthetic poly(A) site. Genes Dev 3: 1019-1025.
10. Luther S A, Lopez T, Bai W, Hanahan D, Cyster J G (2000) BLC expression in pancreatic islets causes B cell recruitment and lymphotoxin-dependent lymphoid neogenesis. Immunity 12: 471-481.
11. Messina J L, Fenstermacher D A, Eschrich S, Qu X, Berglund A E, et al. (2012) 12-Chemokine gene signature identifies lymph node-like structures in melanoma: potential for patient selection for immunotherapy? Sci Rep 2: 765.
12. Meyer H, Damon I K, Esposito J J (2004) Orthopoxvirus diagnostics. Methods Mol Biol 269: 119-134.
13. Milne K, Kobel M, Kalloger S E, Barnes R O, Gao D, et al. (2009) Systematic analysis of immune infiltrates in high-grade serous ovarian cancer reveals CD20, FoxP3 and TIA-1 as positive prognostic factors. PLoS One 4: e6412.
14. Myers E W, Miller W (1988) Optimal alignments in linear space. Comput Appl Biosci 4: 11-17.
15. Neyt K, Perros F, GeurtsvanKessel C H, Hammad H, Lambrecht B N (2012) Tertiary lymphoid organs in infection and autoimmunity. Trends Immunol 33: 297-305.
16. Nielsen J S, Sahota R A, Milne K, Kost S E, Nesslinger N J, et al. (2012) CD20+ tumor-infiltrating lymphocytes have an atypical CD27− memory phenotype and together with CD8+ T cells promote favorable prognosis in ovarian cancer. Clin Cancer Res 18: 3281-3292.
17. Patel M R, Kratzke R A (2013) Oncolytic virus therapy for cancer: the first wave of translational clinical trials. Transl Res 161: 355-364.
18. Peng D, Kryczek I, Nagarsheth N, Zhao L, Wei S, et al. (2015) Epigenetic silencing of TH1-type chemokines shapes tumour immunity and immunotherapy. Nature 527: 249-253.
19. Rintoul J L, Wang J, Gammon D B, van Buuren N J, Garson K, et al. (2011) A selectable and excisable marker system for the rapid creation of recombinant poxviruses. PLoS One 6: e24643.
20. Silina K, Rulle U, Kalnina Z, Line A (2014) Manipulation of tumour-infiltrating B cells and tertiary lymphoid structures: a novel anti-cancer treatment avenue? Cancer Immunol Immunother 63: 643-662.
21. Stojdl D F, Lichty B D, tenOever B R, Paterson J M, Power A T, et al. (2003) VSV strains with defects in their ability to shutdown innate immunity are potent systemic anti-cancer agents. Cancer Cell 4: 263-275.
22. Wall E M, Milne K, Martin M L, Watson P H, Theiss P, et al. (2007) Spontaneous mammary tumors differ widely in their inherent sensitivity to adoptively transferred T cells. Cancer Res 67: 6442-6450.
23. West N R, Milne K, Truong P T, Macpherson N, Nelson B H, et al. (2011) Tumor-infiltrating lymphocytes predict response to anthracycline-based chemotherapy in estrogen receptor-negative breast cancer. Breast Cancer Res 13: R126.
24. Ausubel et al., Current Protocols in Molecular Biology, John Wiley & Sons, New York, N.Y., 1998

All citations are hereby incorporated by reference.

The present invention has been described with regard to one or more embodiments. However, it will be apparent to persons skilled in the art that a number of variations and modifications can be made without departing from the scope of the invention as defined in the claims.

1. A recombinant oncolytic virus comprising a heterologous nucleic acid sequence encoding a B cell attractant polypeptide or a T cell attractant polypeptide wherein the heterologous nucleic acid sequence is stably incorporated into the genome of the recombinant oncolytic virus.

2. The recombinant oncolytic virus of claim 1, wherein the oncolytic virus is attenuated.

3. The recombinant oncolytic virus of claim 1, wherein the oncolytic virus is an oncolytic RNA virus or an oncolytic DNA virus.

4. The recombinant oncolytic virus of claim 1, wherein the oncolytic virus is an oncolytic RNA virus and the heterologous nucleic acid sequence encodes a B cell attractant polypeptide.

5. The recombinant oncolytic RNA virus of claim 3, wherein the oncolytic RNA virus is a vesicular stomatitis virus (VSV), Maraba Virus, Newcastle Disease Virus, Poliovirus, Measles Virus or Reovirus.

6. The recombinant oncolytic virus of claim 1, wherein the oncolytic virus is an oncolytic DNA virus and the heterologous nucleic acid sequence encodes a T cell attractant polypeptide.

7. The recombinant oncolytic DNA virus of claim 3, wherein the oncolytic DNA virus is a Vaccinia Virus (VV), Herpes Simplex Virus (HSV), or Adenovirus.

8. The recombinant oncolytic virus of claim 1, wherein the heterologous nucleic acid sequence encoding a B cell attractant polypeptide is CXCL12 or CXCL13.

9. The recombinant oncolytic virus of claim 1, wherein the heterologous nucleic acid sequence encoding a T cell attractant polypeptide is CXCL10.

10. The recombinant oncolytic virus of claim 1, wherein the oncolytic virus is VSV-CXCL12, VV-CXCL12, VSV-CXCL13, VV-CXCL13, VSV-CXCL10 or VV-CXCL10.

11. A pharmaceutical composition comprising the recombinant oncolytic virus of claim 1 and a pharmaceutically acceptable carrier.

12. The pharmaceutical composition of claim 11 wherein the recombinant oncolytic virus comprises a VSV-CXCL13 in combination with a VV-CXCL12, a VV-CXCL13 or a VV-CXCL10.

13. The pharmaceutical composition of claim 11 wherein the composition is formulated for systemic administration.

14. A method of treating a cancer comprising administering an effective amount of the recombinant oncolytic virus of claim 1, to a subject in need thereof.

15. The method of claim 14 wherein the cancer is a breast cancer, colorectal cancer, lung cancer, melanoma, or ovarian cancer.

16. A method of recruiting immune cells to a tumour comprising contacting the tumour with the recombinant oncolytic virus of claim 1.

17. A method of inhibiting the growth or promoting the killing of a tumour cell, the method comprising contacting the tumour cell with the recombinant oncolytic virus of claim 1.

18. The method of claim 17 wherein the recombinant oncolytic virus is provided at a dosage sufficient to cause cell death of the tumor cell.

19. (canceled)

20. A pharmaceutical composition comprising the recombinant oncolytic virus of claim 4 and a pharmaceutically acceptable carrier.

21. A pharmaceutical composition comprising the recombinant oncolytic virus of claim 6 and a pharmaceutically acceptable carrier.


SEQUENCE LISTING (28 sequences)

SEQ ID NO: 1 - DNA, 365 bp, Mus musculus
gagaagcgcttcatccaccgctgagagacatcccgagccaaccttccggaagcctccccatcagcaccatgaacccaagtgctgccgtcattttctgcctcatcctgctgggtctgagtgggactcaagggatccctctcgcaaggacggtccgctgcaactgcatccatatcgatgacgggccagtgagaatgagggccatagggaagcttgaaatcatccctgcgagcctatcctgcccacgtgttgagatcattgccacgatgaaaaagaatgatgagcagagatgtctgaatccggaatctaagaccatcaagaatttaatgaaagcgtttagccaaaaaaggtctaaaagggctccttaa

SEQ ID NO: 2 - protein, 98 aa, Mus musculus
MNPSAAVIFCLILLGLSGTQGIPLARTVRCNCIHIDDGPVRMRAIGKLEIIPASLSCPRVEIIATMKKNDEQRCLNPESKTIKNLMKAFSQKRSKRAP

SEQ ID NO: 3 - DNA, 1172 bp, Homo sapiens
gagacattcctcaattgcttagacatattctgagcctacagcagaggaacctccagtctcagcaccatgaatcaaactgcgattctgatttgctgccttatctttctgactctaagtggcattcaaggagtacctctctctagaaccgtacgctgtacctgcatcagcattagtaatcaacctgttaatccaaggtctttagaaaaacttgaaattattcctgcaagccaattttgtccacgtgttgagatcattgctacaatgaaaaagaagggtgagaagagatgtctgaatccagaatcgaaggccatcaagaatttactgaaagcagttagcaaggaaatgtctaaaagatctccttaaaaccagaggggagcaaaatcgatgcagtgcttccaaggatggaccacacagaggctgcctctcccatcacttccctacatggagtatatgtcaagccataattgttcttagtttgcagttacactaaaaggtgaccaatgatggtcaccaaatcagctgctactactcctgtaggaaggttaatgttcatcatcctaagctattcagtaataactctaccctggcactataatgtaagctctactgaggtgctatgttcttagtggatgttctgaccctgcttcaaatatttccctcacctttcccatcttccaagggtactaaggaatctttctgctttggggtttatcagaattctcagaatctcaaataactaaaaggtatgcaatcaaatctgctttttaaagaatgctctttacttcatggacttccactgccatcctcccaaggggcccaaattctttcagtggctacctacatacaattccaaacacatacaggaaggtagaaatatctgaaaatgtatgtgtaagtattcttatttaatgaaagactgtacaaagtataagtcttagatgtatatatttcctatattgttttcagtgtacatggaataacatgtaattaagtactatgtatcaatgagtaacaggaaaattttaaaaatacagatagatatatgctctgcatgttacataagataaatgtgctgaatggttttcaaataaaaatgaggtactctcctggaaatattaagaaagactatctaaatgttgaaagatcaaaaggttaataaagtaattataact

SEQ ID NO: 4 - protein, 98 aa, Homo sapiens
MNQTAILICCLIFLTLSGIQGVPLSRTVRCTCISISNQPVNPRSLEKLEIIPASQFCPRVEIIATMKKKGEKRCLNPESKAIKNLLKAVSKERSKRSP

SEQ ID NO: 5 - DNA, 362 bp, Mus musculus
gagctaaaggttgaactccacctccaggcagaatgaggctcagcacagcaacgctgcttctcctcctggccagctgcctctctccaggccacggtattctggaagcccattacacaaacttaaaatgtaggtgttctggagtgatttcaactgttgtcggtctaaacatcatagatcggattcaagttacgccccctgggaatggctgccccaaaactgaagttgtgatctggaccaagatgaagaaagttatatgtgtgaatcctcgtgccaaatggttacaaagattattaagacatgtccaaagcaaaagtctgtcttcaactccccaagctccagtgagtaagagaagagctgcctga

SEQ ID NO: 6 - protein, 109 aa, Mus musculus
MRLSTATLLLLLASCLSPGHGILEAHYTNLKCRCSGVISTVVGLNIIDRIQVTPPGNGCPKTEVVIWTKMKKVICVNPRAKWLQRLLRHVQSKSLSSTPQAPVSKRRAA

SEQ ID NO: 7 - DNA, 1219 bp, Homo sapiens
gagaagatgtttgaaaaaactgactctgctaatgagcctggactcagagctcaagtctgaactctacctccagacagaatgaagttcatctcgacatctctgcttctcatgctgctggtcagcagcctctctccagtccaaggtgttctggaggtctattacacaagcttgaggtgtagatgtgtccaagagagctcagtctttatccctagacgcttcattgatcgaattcaaatcttgccccgtgggaatggttgtccaagaaaagaaatcatagtctggaagaagaacaagtcaattgtgtgtgtggaccctcaagctgaatggatacaaagaatgatggaagtattgagaaaaagaagttcttcaactctaccagttccagtgtttaagagaaagattccctgatgctgatatttccactaagaacacctgcattcttcccttatccctgctctggattttagttttgtgcttagttaaatcttttccaggaaaaagaacttccccatacaaataagcatgagactatgtaaaaataaccttgcagaagctgatggggcaaactcaagcttcttcactcacagcaccctatatacacttggagtttgcattcttattcatcagggaggaaagtttctttgaaaatagttattcagttataagtaatacaggattattttgattatatacttgttgtttaatgtttaaaatttcttagaaaacaatggaatgagaatttaagcctcaaatttgaacatgtggcttgaattaagaagaaaattatggcatatattaaaagcaggcttctatgaaagactcaaaaagctgcctgggaggcagatggaacttgagcctgtcaagaggcaaaggaatccatgtagtagatatcctctgcttaaaaactcactacggaggagaattaagtcctacttttaaagaatttctttataaaatttactgtctaagattaatagcattcgaagatccccagacttcatagaatactcagggaaagcatttaaagggtgatgtacacatgtatcctttcacacatttgccttgacaaacttctttcactcacatctttttcactgactttttttgtggggggcggggccggggggactctggtatctaattctttaatgattcctataaatctaatgacattcaataaagttgagcaaacattttacttaaaaaaaaaaaaaaaaaa

SEQ ID NO: 8 - protein, 109 aa, Homo sapiens
MKFISTSLLLMLLVSSLSPVQGVLEVYYTSLRCRCVQESSVFIPRRFIDRIQILPRGNGCPRKEIIVWKKNKSIVCVDPQAEWIQRMMEVLRKRSSSTLPVPVFKRKIP

SEQ ID NO: 9 - DNA, 5627 bp, Mus musculus
gcactttcactctcggtccacctcggtgtcctcttgctgtccagctctgcagcctccggcgcgccctcccgcccacgccatggacgccaaggtcgtcgccgtgctggccctggtgctggccgcgctctgcatcagtgacggtaaaccagtcagcctgagctaccgatgcccctgccggttcttcgagagccacatcgccagagccaacgtcaagcatctgaaaatcctcaacactccaaactgtgcccttcagattgttgcacggctgaagaacaacaacagacaagtgtgcattgacccgaaattaaagtggatccaagagtacctggagaaagctttaaacaaggggcgcagagaagaaaaagtggggaaaaaagaaaagataggaaaaaagaagcgacagaagaagagaaaggctgcccagaaaaggaaaaactagttccccgcttcctgccgatggaccgcagtgcgctctgctctggcgctttgtaactcgctcctccctcttcgggggcagaccccacactccgggcaggtgctcaaacctgacggtaaactcttccctcttcgggggcagaccccataccccggggcgggtgcttaggctttcctgccctggtggccacaccagctgctgtatttatgtgcttcataaggccctgctctgtctgctaaagctatgaagaaagatgtgcagagactggggtggagactaagccaaagaggagctgcctagcctggcagcattgccccgagctgagcccccttggccaggacttcacaaggctcacacctacaatcccatgaaggccagggtggtctgcttagccaggaaagggcaagtgccttcccctcggccacactgccccttgtggccttctcgggacatgtggtaactgacttgctctcaggcccacccgcagcttttccaaatacctgcagccttcagccctgctgccctgcctgtgggagcagctttgactccagtccagaagggtttctgcagactgtgttgggtgagacgcagaaaggatgaaatctcagaacacatgtcagctgcttctcaggaaatcttttctttggacaattcactttagagtctttaaacgggtctctcgtggggaggatagatgtgctctggaactttctgaaggaccagcagcttcagggactcttagtctgtccttccccacttttggtcccaacatccctgggatggtgtgctgtctgggcaccacggtctccatcctcactcctgagagatttctgccttctgtgagttgggttaaagctctggaattatctactatcccaatccactaccctcacctggcaatatttgtctgtttttgtttgtttgtttgtttgtttttgtcttttgccagtttgaattagaaggcaaggctctgattttagtagtgttttggaaaaggacttttttcttcaccttcctctttgcctcatgtgtacacacacacacacatcttgtaccccagacctctgggtataattttcataattggtgcagaaagaagaaatgatctgaagatgtgttaaatggattgcaggggaaggaaggcccagggccctgtgtgtcatgccctcttgggttcctaagttctatgttccttagaggttctagcattaaacagataaagcccttcatggtcctggctgaggaagagtcttgctagggggattcagggaagacccgtgttaccagctcttaccctttatctggacagctctcctaccctgtatcttctcctcagatctgaggatagcaggctggactattggtgggcacctttcaagcccagggctactgtttgtcctgtggcagccggctacagtctcgtctgagtggcctcatctggacccttcctgttattaataaaacgcttctggaggccagatctgtgctcaagccatagttctgcttagaaagggatgccccacccttaccggacactgggaagaactgttggcccctagaaaccaaaggccaaactgaggctgccctgagttggaagaccactttctgaaatgcccatggactctgcctcccaaccattcgtctctcactcctagcagagctgtctgtgcagactgtttcttaggaggcacagcaagctccagggaaccctctgtgcttatgaagctcgtctggtgggcaaccccagcccactggacagagtcctcatggaaatgcctgggaagctgatttcatctaaggatgggttgaagtaggatgtgctcctgcgacttctcaggcaggtgagaggggtagtccttacactgtctagcataaacgccttccggaaggacctgcagctccagagaccacctcctgagcaccaagacctcttctggtggtgtggaaccagccaagagatttcaaggaagagtgattatttgatgaatgctatgggaatggcctcttctcttggagttctgaggcctggggatgcccaggaacactgggcacctgctgctgttagggccaatgcatagtctcagcaccggtgtcctaaggttaaggcggtgcgccttgtcatgtgctccttgtaccatgccatctgtgccagtgtgtgtctgcctcaccctgtgcttgacatgttcacccatcttctctgcttcccgccaccatccagatcctcagcggccgccccggctgtgcccttccctgctctcccgctctctcaggcctcggaaggaagatcggtggctgcgagctgaactaaggagtagggcctgtggctcagcgctaggccacgcacgcagcatcccaggcatgtggtgagaaactgccttaatgtgtctcctctgttcttgtcaacaggaggctcaagatgtgagaggtgtgagtcagacgcccgaggaacttacaggaggagcctaggtctgaagtcagtgttagggaagggcccatagccacttcctctgctcctgagcagggctgaagccgtttgcaagggacttgctttgcacagttttgctgtactttcacattttattatgtagcaagatacatggtgatttttttttttttcatttagcctgattttccaacgtcattggtgacaggccaaggccactatgttatttcctttgttctggtatccttcccttggaggaccttctctgagtagtggctccccaggtttgtcctttgagctgaggcaggaggctcacccattcttctgaataggaactgggtgttcccaccccccaaggactgcagggctttcccaagctgaggcaggaacgtgaggccagggaagagtgagcttcaccctcatcccacgctgtcctcctcaacccaccatgctcatcattctgtctcatccatccatccatccatccattcatcgccatgtgtccgcaagactgtctccatgaccctgaaaaaggactctcgagatgaaatcctttattcaaatgggacagcaagaaggaaaagccaatgtctggtgtctctccccccgcccctaccctgcgcgcatctatgtcttgtttggaatattgtctcttcaaccccctgttcatgtccttctcactcatgatcgatgtcttgtctgtgcactgtctctaacccaaatgcaaaggctgagtgtgaggtgatggccccgaggtccaggttgtagtcatggaaagagccctgctgtctcccttctcagggggcccattttagacacacaaagcccaaagaaaggtggtttgcaacagtgcttagctcgagcctccatatttccataactgttagcttaaaactgtggggttttaccttcctggaaccaaatgcattcttctgttgaggagtaacaggtctcaattcttttcaattaattttaaaagtcaatcactaagagcatcggctttgggccctgatgggcaggcatttccctggaaagggggtgaactacctacctctcctcaagacagccgaagggtgggattggtgccgctctgggaagcgtggccccaggagttttgtcctctgcagtttttaatgcaagttcactgccactttgacaaaagcccaattagaagccagtctctagttccttaaacaaaacagacagagtaaggaaaggaaggagggtggcagccagctggctggacactcgagaaagacggggaagtaagctacagaaagatagtcttcaaaaacaggtgtttgagagtgaatactctgtagaattgttagtggggtgtgtgtggtggtggtggggggatttctacaaaatagtcctttaagttgagtttacagcagatgaaaaatccaaccagcaaaattttgatcaaatttgaacaaaaacccaaaaacctaaaactgttgagcaggttgcgatgaggagcacagggctagctgcagagctggatcctcaggaggatagcgaattattttcaaccctggaatagaaaccacacactggcttgctgtgcaccagccactttgcatctaatccaagctttgaagggtgttgcttgggaggaaacaaatacagccttccatcttcactccagttagggatcctttcaaagtctccttcacagtgaggaaaaagagaagggtagaaactttagggagccggatttgtgtatcaattcctccgctgacagtcagtttctagatggagacagcctgcttaaagcaaatccgaatttaaataggacatttacatcggaaaagtctctccctaccttaatcccccattctcttgctttcaaaatacaagcacagcagtccttgaatggctgttgacccagggcacctagctgtccctgctggtcctggggctgccagaattcccttgggcgccaagcaacctgccaggtagccagtccctctgttacaagcctttgcatctggatagggaaaggggtggagacatacagtctgctttgtgttgaaacccagatttgtaccctgtgtttatacactgctgctggctcccgaggacagtgggactttagcaaggaagtgcagccgaggggtaaagagccctctggttcattgcctgatcggctttgagagagggtttggagggcaaggggctgcattcctctgagggacttggcctgaggcctttcgggcctctccagtgggttctgtttatcctctcatgggtgattatctcagtggtgtcaccaggggcttcctcccagaagtcagtcatccccaggccgtgcacccttttcagctggatgagagccagggatgcattctctccaaacagctaccctggcccattttaaggtaatctcattcttcaaaatgttccatagaatcctccaaattcccccagcagacttctaccctcgccaagttcccaaaacccactcagcaaagttgccaacctcgacgggctagcagtgtctaagcagcgatgggttcagtgttgtgtgtggtgaatactgtattttgtttcagttctgtctcccagataatgtgaaaacggtccaggagaaggcagcttcctatatgcagcgtgtgctttcttattcttatttttaatatatgacagttatttgagaagccatttctactttgaagtcattatcgatgaaagtgatgtatcttcacctaccattttcctaataaagttctgtattcaaatata

SEQ ID NO: 10 - protein, 119 aa, Mus musculus
MDAKVVAVLALVLAALCISDGKPVSLSYRCPCRFFESHIARANVKHLKILNTPNCALQIVARLKNNNRQVCIDPKLKWIQEYLEKALNKGRREEKVGKKEKIGKKKRQKKRKAAQKRKN

SEQ ID NO: 11 - DNA, 3549 bp, Homo sapiens
gccgcactttcactctccgtcagccgcattgcccgctcggcgtccggcccccgacccgcgctcgtccgcccgcccgcccgcccgcccgcgccatgaacgccaaggtcgtggtcgtgctggtcctcgtgctgaccgcgctctgcctcagcgacgggaagcccgtcagcctgagctacagatgcccatgccgattcttcgaaagccatgttgccagagccaacgtcaagcatctcaaaattctcaacactccaaactgtgcccttcagattgtagcccggctgaagaacaacaacagacaagtgtgcattgacccgaagctaaagtggattcaggagtacctggagaaagctttaaacaagaggttcaagatgtgagagggtcagacgcctgaggaacccttacagtaggagcccagctctgaaaccagtgttagggaagggcctgccacagcctcccctgccagggcagggccccaggcattgccaagggctttgttttgcacactttgccatattttcaccatttgattatgtagcaaaatacatgacatttatttttcatttagtttgattattcagtgtcactggcgacacgtagcagcttagactaaggccattattgtacttgccttattagagtgtctttccacggagccactcctctgactcagggctcctgggttttgtattctctgagctgtgcaggtggggagactgggctgagggagcctggccccatggtcagccctagggtggagagccaccaagagggacgcctgggggtgccaggaccagtcaacctgggcaaagcctagtgaaggcttctctctgtgggatgggatggtggagggccacatgggaggctcacccccttctccatccacatgggagccgggtctgcctcttctgggagggcagcagggctaccctgagctgaggcagcagtgtgaggccagggcagagtgagacccagccctcatcccgagcacctccacatcctccacgttctgctcatcattctctgtctcatccatcatcatgtgtgtccacgactgtctccatggccccgcaaaaggactctcaggaccaaagctttcatgtaaactgtgcaccaagcaggaaatgaaaatgtcttgtgttacctgaaaacactgtgcacatctgtgtcttgtttggaatattgtccattgtccaatcctatgtttttgttcaaagccagcgtcctcctctgtgaccaatgtcttgatgcatgcactgttccccctgtgcagccgctgagcgaggagatgctccttgggccctttgagtgcagtcctgatcagagccgtggtcctttggggtgaactaccttggttcccccactgatcacaaaaacatggtgggtccatgggcagagcccaagggaattcggtgtgcaccagggttgaccccagaggattgctgccccatcagtgctccctcacatgtcagtaccttcaaactagggccaagcccagcactgcttgaggaaaacaagcattcacaacttgtttttggtttttaaaacccagtccacaaaataaccaatcctggacatgaagattctttcccaattcacatctaacctcatcttcttcaccatttggcaatgccatcatctcctgccttcctcctgggccctctctgctctgcgtgtcacctgtgcttcgggcccttcccacaggacatttctctaagagaacaatgtgctatgtgaagagtaagtcaacctgcctgacatttggagtgttccccttccactgagggcagtcgatagagctgtattaagccacttaaaatgttcacttttgacaaaggcaagcacttgtgggtttttgttttgtttttcattcagtcttacgaatacttttgccctttgattaaagactccagttaaaaaaaattttaatgaagaaagtggaaaacaaggaagtcaaagcaaggaaactatgtaacatgtaggaagtaggaagtaaattatagtgatgtaatcttgaattgtaactgttcttgaatttaataatctgtagggtaattagtaacatgtgttaagtattttcataagtatttcaaattggagcttcatggcagaaggcaaacccatcaacaaaaattgtcccttaaacaaaaattaaaatcctcaatccagctatgttatattgaaaaaatagagcctgagggatctttactagttataaagatacagaactctttcaaaaccttttgaaattaacctctcactataccagtataattgagttttcagtggggcagtcattatccaggtaatccaagatattttaaaatctgtcacgtagaacttggatgtacctgcccccaatccatgaaccaagaccattgaattcttggttgaggaaacaaacatgaccctaaatcttgactacagtcaggaaaggaatcatttctatttctcctccatgggagaaaatagataagagtagaaactgcagggaaaattatttgcataacaattcctctactaacaatcagctccttcctggagactgcccagctaaagcaatatgcatttaaatacagtcttccatttgcaagggaaaagtctcttgtaatccgaatctctttttgctttcgaactgctagtcaagtgcgtccacgagctgtttactagggatccctcatctgtccctccgggacctggtgctgcctctacctgacactcccttgggctccctgtaacctcttcagaggccctcgctgccagctctgtatcaggacccagaggaaggggccagaggctcgttgactggctgtgtgttgggattgagtctgtgccacgtgtttgtgctgtggtgtgtccccctctgtccaggcactgagataccagcgaggaggctccagagggcactctgcttgttattagagattacctcctgagaaaaaaggttccgcttggagcagaggggctgaatagcagaaggttgcacctcccccaaccttagatgttctaagtctttccattggatctcattggacccttccatggtgtgatcgtctgactggtgttatcaccgtgggctccctgactgggagttgatcgcctttcccaggtgctacacccttttccagctggatgagaatttgagtgctctgatccctctacagagcttccctgactcattctgaaggagccccattcctgggaaatattccctagaaacttccaaatcccctaagcagaccactgataaaaccatgtagaaaatttgttattttgcaacctcgctggactctcagtctctgagcagtgaatgattcagtgttaaatgtgatgaatactgtattttgtattgtttcaattgcatctcccagataatgtgaaaatggtccaggagaaggccaattcctatacgcagcgtgctttaaaaaataaataagaaacaactctttgagaaacaacaatttctactttgaagtcataccaatgaaaaaatgtatatgcacttataattttcctaataaagttctgtactcaaatgtagccaccaacagt

SEQ ID NO: 12 - DNA, 3560 bp, Homo sapiens
gccgcactttcactctccgtcagccgcattgcccgctcggcgtccggcccccgacccgcgctcgtccgcccgcccgcccgcccgcccgcgccatgaacgccaaggtcgtggtcgtgctggtcctcgtgctgaccgcgctctgcctcagcgacgggaagcccgtcagcctgagctacagatgcccatgccgattcttcgaaagccatgttgccagagccaacgtcaagcatctcaaaattctcaacactccaaactgtgcccttcagattgtagcccggctgaagaacaacaacagacaagtgtgcattgacccgaagctaaagtggattcaggagtacctggagaaagctttaaacaagaggttcaagatgtgagagggtcagacgcctgaggaacccttacagtaggagcccagctctgaaaccagtgttagggaagggcctgccacagcctcccctgccagggcagggccccaggcattgccaagggctttgttttgcacactttgccatattttcaccatttgattatgtagcaaaatacatgacatttatttttcatttagtttgattattcagtgtcactggcgacacgtagcagcttagactaaggccattattgtacttgccttattagagtgtctttccacggagccactcctctgactcagggctcctgggttttgtattctctgagctgtgcaggtggggagactgggctgagggagcctggccccatggtcagccctagggtggagagccaccaagagggacgcctgggggtgccaggaccagtcaacctgggcaaagcctagtgaaggcttctctctgtgggatgggatggtggagggccacatgggaggctcacccccttctccatccacatgggagccgggtctgcctcttctgggagggcagcagggctaccctgagctgaggcagcagtgtgaggccagggcagagtgagacccagccctcatcccgagcacctccacatcctccacgttctgctcatcattctctgtctcatccatcatcatgtgtgtccacgactgtctccatggccccgcaaaaggactctcaggaccaaagctttcatgtaaactgtgcaccaagcaggaaatgaaaatgtcttgtgttacctgaaaacactgtgcacatctgtgtcttgtttggaatattgtccattgtccaatcctatgtttttgttcaaagccagcgtcctcctctgtgaccaatgtcttgatgcatgcactgttccccctgtgcagccgctgagcgaggagatgctccttgggccctttgagtgcagtcctgatcagagccgtggtcctttggggtgaactaccttggttcccccactgatcacaaaaacatggtgggtccatgggcagagcccaagggaattcggtgtgcaccagggttgaccccagaggattgctgccccatcagtgctccctcacatgtcagtaccttcaaactagggccaagcccagcactgcttgaggaaaacaagcattcacaacttgtttttggtttttaaaacccagtccacaaaataaccaatcctggacatgaagattctttcccaattcacatctaacctcatcttcttcaccatttggcaatgccatcatctcctgccttcctcctgggccctctctgctctgcgtgtcacctgtgcttcgggcccttcccacaggacatttctctaagagaacaatgtgctatgtgaagagtaagtcaacctgcctgacatttggagtgttccccttccactgagggcagtcgatagagctgtattaagccacttaaaatgttcacttttgacaaaggcaagcacttgtgggtttttgttttgtttttcattcagtcttacgaatacttttgccctttgattaaagactccagttaaaaaaaattttaatgaagaaagtggaaaacaaggaagtcaaagcaaggaaactatgtaacatgtaggaagtaggaagtaaattatagtgatgtaatcttgaattgtaactgttcttgaatttaataatctgtagggtaattagtaacatgtgttaagtattttcataagtatttcaaattggagcttcatggcagaaggcaaacccatcaacaaaaattgtcccttaaacaaaaattaaaatcctcaatccagctatgttatattgaaaaaatagagcctgagggatctttactagttataaagatacagaactctttcaaaaccttttgaaattaacctctcactataccagtataattgagttttcagtggggcagtcattatccaggtaatccaagatattttaaaatctgtcacgtagaacttggatgtacctgcccccaatccatgaaccaagaccattgaattcttggttgaggaaacaaacatgaccctaaatcttgactacagtcaggaaaggaatcatttctatttctcctccatgggagaaaatagataagagtagaaactgcagggaaaattatttgcataacaattcctctactaacaatcagctccttcctggagactgcccagctaaagcaatatgcatttaaatacagtcttccatttgcaagggaaaagtctcttgtaatccgaatctctttttgctttcgaactgctagtcaagtgcgtccacgagctgtttactagggatccctcatctgtccctccgggacctggtgctgcctctacctgacactcccttgggctccctgtaacctcttcagaggccctcgctgccagctctgtatcaggacccagaggaaggggccagaggctcgttgactggctgtgtgttgggattgagtctgtgccacgtgtttgtgctgtggtgtgtccccctctgtccaggcactgagataccagcgaggaggctccagagggcactctgcttgttattagagattacctcctgagaaaaaaggttccgcttggagcagaggggctgaatagcagaaggttgcacctcccccaaccttagatgttctaagtctttccattggatctcattggacccttccatggtgtgatcgtctgactggtgttatcaccgtgggctccctgactgggagttgatcgcctttcccaggtgctacacccttttccagctggatgagaatttgagtgctctgatccctctacagagcttccctgactcattctgaaggagccccattcctgggaaatattccctagaaacttccaaatcccctaagcagaccactgataaaaccatgtagaaaatttgttattttgcaacctcgctggactctcagtctctgagcagtgaatgattcagtgttaaatgtgatgaatactgtattttgtattgtttcaattgcatctcccagataatgtgaaaatggtccaggagaaggccaattcctatacgcagcgtgctttaaaaaataaataagaaacaactctttgagaaacaacaatttctactttgaagtcataccaatgaaaaaatgtatatgcacttataattttcctaataaagttctgtactcaaatgtagccaccaaaaaaaaaaaaaaaaa

SEQ ID NO: 13 - protein, 93 aa, Homo sapiens
MNAKVVVVLVLVLTALCLSDGKPVSLSYRCPCRFFESHVARANVKHLKILNTPNCALQIVARLKNNNRQVCIDPKLKWIQEYLEKALNKRFKM

SEQ ID NO: 14 - protein, 89 aa, Homo sapiens
MNAKVVVVLVLVLTALCLSDGKPVSLSYRCPCRFFESHVARANVKHLKILNTPNCALQIVARLKNNNRQVCIDPKLKWIQEYLEKALNK

SEQ ID NO: 15 - protein, 119 aa, Homo sapiens
MNAKVVVVLVLVLTALCLSDGKPVSLSYRCPCRFFESHVARANVKHLKILNTPNCALQIVARLKNNNRQVCIDPKLKWIQEYLEKALNKGRREEKVGKKEKIGKKKRQKKRKAAQKRKN

SEQ ID NO: 16 - protein, 140 aa, Homo sapiens
MNAKVVVVLVLVLTALCLSDGKPVSLSYRCPCRFFESHVARANVKHLKILNTPNCALQIVARLKNNNRQVCIDPKLKWIQEYLEKALNNLISAAPAGKRVIAGARALHPSPPRACPTARALCEIRLWPPPEWSWPSPGDV

SEQ ID NO: 17 - protein, 103 aa, Homo sapiens
MNAKVVVVLVLVLTALCLSDGKPVSLSYRCPCRFFESHYCTCLIRVSFHGATPLTQGSWVLYSLSCAGGETGLREPGPMVSPRVESHQEGRLGVPGPVNLGKA

SEQ ID NO: 18 - DNA, 40 bp, Artificial Sequence
  synthetic Vaccinia Virus early/late promoter
aaaaattgaaattttattttttttttttggaatataaata

SEQ ID NO: 19 - DNA, 33 bp, Artificial Sequence
  Primer VSV_CXCL13_left
taagcactcgaggagctaaaggttgaactccac

SEQ ID NO: 20 - DNA, 33 bp, Artificial Sequence
  Primer VSV_CXCL13_right
tgcttagctagctcaggcagctcttctcttact

SEQ ID NO: 21 - DNA, 32 bp, Artificial Sequence
  Primer VSV_CXCL10_left
taagcactcgaggagaagcgcttcatccaccg

SEQ ID NO: 22 - DNA, 33 bp, Artificial Sequence
  Primer VSV_CXCL10_right
tgcttagctagcttaaggagcccttttagacct

SEQ ID NO: 23 - DNA, 73 bp, Artificial Sequence
  Primer VV_CXCL13_left
gatccaactagtaaaaattgaaattttattttttttttttggaatataaatagagctaaaggttgaactccac

SEQ ID NO: 24 - DNA, 33 bp, Artificial Sequence
  Primer VV_CXCL13_right
tgcttaactagttcaggcagctcttctcttact

SEQ ID NO: 25 - DNA, 72 bp, Artificial Sequence
  Primer VV_CXCL10_left
gatccaactagtaaaaattgaaattttattttttttttttggaatataaatagagaagcgcttcatccaccg

SEQ ID NO: 26 - DNA, 33 bp, Artificial Sequence
  Primer VV_CXCL10_right
tgcttaactagtttaaggagcccttttagacct

SEQ ID NO: 27 - DNA, 22 bp, Artificial Sequence
  Primer VV_TK_Fwd
atgaacggcggacatattcagt

SEQ ID NO: 28 - DNA, 22 bp, Artificial Sequence
  Primer VV_TK_Rev
gagtcgatgtaacactttctac